(19) United States
(12) Patent Application Publication
(10) Pub. No.: US 2026/0108733 A1
(43) Pub. Date: Apr. 23, 2026

(54) SYSTEM AND METHOD FOR ELECTRIC FIELD TRANSMISSION

(71) Applicant: FIELDCURE CO., LTD., Seoul (KR)

(72) Inventors: Jin Young HONG, Seoul (KR); Dong Jun LEE, Seoul (KR); Woo Jung KANG, Yonginsi (KR); Jong Hyun KIM, Seoul (KR)

(73) Assignee: FIELDCURE CO., LTD., Seoul (KR)

(21) Appl. No.: 19/117,658

(22) PCT Filed: Oct. 24, 2023
(86) PCT No.: PCT/KR2023/016520

§ 371 Date: Apr. 2, 2025

(30) Foreign Application Priority Data
Oct. 25, 2022 (KR) 10-2022-0137968
Oct. 23, 2023 (KR) 10-2023-0141860

(51) Int. Cl. A61N 1/36; A61N 1/02; A61N 1/04

(52) U.S. Cl. CPC A61N 1/36002; A61N 1/025; A61N 1/0408; A61N 1/0476; A61N 1/36034

(57) ABSTRACT

An embodiment of the present invention relates to an electric field transmission system which uses an electrode array comprising multiple individual electrodes to transmit an electric field to a region of interest within a three-dimensional object. The electric field transmission system comprises: multiple generators for providing an electric field; multiple electrode sub-arrays for transmitting alternating current power generated from the multiple generators to an object, each electrode sub-array comprising some of the multiple individual electrodes included in the electrode array; a switching router for connecting the multiple generators to the multiple electrode sub-arrays one-to-one; and a control unit for controlling the multiple generators, the multiple electrode sub-arrays, and the switching router. Therefore, there is an effect of independently controlling the voltage or current of individual electrodes.

### CROSS-REFERENCE TO RELATED APPLICATION

This application is an International Application No. PCT/KR2023/016520 filed on Oct. 24, 2023, which claims priority from Korean Patent Application No. 10-2023-0141860 filed on Oct. 23, 2023 and Korean Provisional Patent Application No. 10-2022-0137968 filed on Oct. 25, 2022, the entire contents of which are incorporated herein by reference.

### TECHNICAL FIELD

The present disclosure relates to a system and a method of delivering an electric field, and more particularly, to a system and a method of delivering an electric field which delivers an electric field to a region of interest in a 3-dimensional subject.

### BACKGROUND ART

High frequency therapy is a medical approach that applies energy to the human body using alternating current power (voltage or current) and has the objective of treating diseases and improving symptoms. It uses energy to regulate blood flow, metabolic activity and inflammation in tissues thereby alleviating or treating various symptoms.

For example, the hyperthermia for cancer cells uses the principle of applying high temperature heat energy to cancer tissue to damage cancer cells. Cancer cells are more sensitive to heat than normal cells, and when exposed to relatively high temperatures, the physiological processes inside the cells may be abnormal or the cell membrane may be damaged to result in apoptosis, and thrombi may be formed to decrease blood flow, which can lead to necrosis due to the blockage of nutrient supply.

The currently commercialized tumor treating fields (TTFields) are a proven cancer treatment that has been approved by the FDA for relapsed glioblastoma and newly diagnosed glioblastoma.

A system for tumor treating fields, or a system for delivering a therapeutic electric field to a target region of a subject, comprises a generator (alternating current signal generator), a plurality of electrode arrays (electrode pads or transducer array), and a divider that connects the generator to a pair of electrode arrays.

According to U.S. Pat. Nos. 8,715,203 or 8,764,675, each electrode array consists of a plurality of individual electrodes that are attached to the surface of the subject. Here, the individual electrodes are connected in parallel to the wires for the current path that connects to the electrode array. In other words, the individual electrode has an isopotential surface that shares the same voltage. The typical individual electrode is a capacitive coupled electrode formed of a cylindrical dielectric (ceramic). Each electrode is sandwiched between an electrically conductive medical gel (hydrogel) and an adhesive tape to apply pressure. The medical gel provides an electrical connection between the hard electrode and the skin along the patient's contour. A typical electrode array for glioblastoma consists of nine individual electrodes with a diameter of 1.8 cm in a 3×3 matrix structure in which a temperature sensor is in thermal contact with the individual electrode to measure the temperature between the electrode and the skin.

The generator is connected via the divider to two pairs of electrode arrays (a pair of first electrode arrays and a pair of second electrode arrays) to cover two directions orthogonal to each other.

The system for delivering an electric field connects the first pair of electrode arrays for first time to flow electric current and selects a second pair of electrode arrays for next second time to flow electric current. This cyclical operation continues during the treatment. The alternating current generated by the generator is connected to a pair of selected electrode arrays (the first electrode array and the second electrode array) via the divider, and flows along the current path consisting of the electrodes of the first electrode array and the skin of the subject to which each electrode is attached, and the body of the subject, the skin of the subject to which the individual electrode of the second electrode array is attached, and the electrodes of the second electrode array. At this time, a therapeutic electric field of a certain intensity (1 V/cm) or more is delivered to the target region of the cancer cell, and this therapeutic electric field destroys the cancer cell by interfering with or delaying the division of the cancer cell.

When the axis of cancer cell division and the direction of the electric field coincide, the therapeutic effect is greater, however if the axis and the direction are perpendicular to each other, the therapeutic effect disappears. Since cancer cells divide in all directions, two pairs of electrode arrays are placed orthogonally to each other and alternately deliver the therapeutic electric field. The frequency of the alternating current is in the range of 10˜1 MHz, and the optimal frequency is determined according to the type and size of the target tumor cells. When the therapeutic electric field is delivered to the target region by alternating the first electrode array pairs and the second electrode array pairs via the divider, if the temperature of the individual electrode rises above the limit temperature (41 degrees Celsius), the treatment is stopped until the temperature is below the limit temperature again.

However, due to the edge effect of the electrode array, input impedance of some individual electrodes located at the vertex or boundary may be lower, and this will inevitably lead to an increase in the temperature of the skin, the risk of skin burns and a decrease in the therapeutic effect if the intensity of the current flowing to a particular individual electrode rises by more than a certain intensity. This can lead to problems that reduce the effectiveness of treatment and reduce safety.

Therefore, in order to increase treatment effectiveness, increase safety, and reduce risk, system that can control the generator, the divider, and the electrode array more efficiently are needed.

### DISCLOSURE OF INVENTION

### Technical Problem

In order to solve the problem, the present disclosure provides a system and a method of delivering an electric field capable of independently controlling voltage or current of individual electrodes.

### Solution to Problem

A system for delivering an electric field according to an embodiment of the inventive concept in order to accomplish the aforementioned object is a system for delivering an electric field which delivers the electric field to a region of interest in a 3D subject using an electrode array including a plurality of individual electrodes, comprising: a plurality of generators providing the electric field; a plurality of electrode sub-arrays delivering alternating current power, generated by the plurality of generators, to the subject, the electrode sub-array including some of the individual electrodes among the plurality of individual electrodes in the electrode array; a switching router one-to-one connecting the plurality of generators and the plurality of electrode sub-arrays; and a controller controlling the plurality of generators, the plurality of electrode sub-arrays and the switching router.

Here, the controller may provide an independently adjustable current to the electrode sub-array to prevent excessive current from being applied to a particular individual electrode.

Here, the controller may independently control a magnitude of voltage or current and independently control duty cycle of applying voltage or current, for each of the plurality of the generators.

Here, the controller is configured to determine a mapping method between the plurality of generators and the plurality of electrode sub-arrays, and control the switching router according to the mapping method.

Here, the switching router is configured to connect the plurality of generators and the plurality of sub-arrays to be mapped.

Here, the switching router and the electrode switch may include a relay switch.

Here, the switching router and the electrode switch may further comprise a field-effect transistor (FET) connected to the relay switch in series.

Here, a temperature sensor detecting temperature rise of each individual electrode may be further included, and the controller is configured to apply current to each electrode sub-array where a temperature rise is detected by the temperature sensor by independently reducing the duty cycle of applying current, or apply voltage and current of the generator connected to each electrode sub-array to each electrode sub-array where a temperature rise is detected by independently decreasing the intensity of voltage and current.

Here, the controller may receive electrode layout information from an external treatment planning system or an external transducer array layout system, and the electrode layout information may include the position of the electrode array, the selection of the electrode sub-array, and the voltage or current intensity for the electrode sub-array.

Here, the position of the electrode array contained in the electrode layout information may be a position of the electrode array represented in a 3D model of the subject.

Here, the controller is configured to determine a mapping method between the plurality of generators and the plurality of electrode sub-arrays according to the received electrode layout information, and control the switching router according to the mapping method.

Here, according to the received electrode layout information, the controller may set a connection order of the electrode sub-arrays and a duration of operation, and determine the mapping method between the plurality of generators and the electrode sub-arrays in active and inactive by each mode of operation, and an initial value of voltage or current of each generator.

A system for delivering an electric field according to an embodiment of the inventive concept, which is designed to accomplish the aforementioned object, is a system for delivering tumor treating fields (TTFields) to a region of interest in a 3D subject using an electrode array comprising a plurality of individual electrodes. The system comprises: a generator for providing the electric field; an electrode sub-array for delivering alternating current power, generated by the generator, to the subject, the electrode sub-array including some of the individual electrodes among the plurality of individual electrodes in the electrode array; a switching router connecting the generator and the electrode sub-array; at least one electrode switch connecting the electrode sub-array and at least one of the individual electrodes; and a controller for controlling the generator, the electrode sub-array, and the switching router.

Here, the controller is configured to select at least one electrode sub-array and provide an adjustable current in order to ensure that the dose distribution delivered to the region of interest of the subject corresponds to the location, shape, and size of the region of interest.

Here, the controller is configured to provide an independently adjustable current to the electrode sub-array to prevent excessive current from being applied to a particular individual electrode.

Here, the controller may independently control a magnitude of voltage or current and independently control a duty cycle of applying voltage or current, for the generator.

Here, the switching router and the electrode switch may include a relay switch.

Additionally, a field-effect transistor (FET) may be connected in series with the relay switch.

Here, each of the individual electrodes in the electrode sub-array may further include a temperature sensor detecting temperature rise, and the controller may apply current to an electrode sub-array where a temperature rise is detected by independently reducing the duty cycle, or may reduce the voltage and current intensity of the generator connected to that electrode sub-array.

Here, the controller may receive electrode operation information including i) whether to select a connection, ii) a duration of operation and iii) an operation power for the electrode sub-array and a plurality of external electrode sub-arrays from an external transducer array layout system, to sequentially and repeatedly implement the connection selection and the application of the operation power for each electrode sub-array and the plurality of external electrode sub-arrays based on the preset duration of operation.

A method of delivering an electric field according to an embodiment of the inventive concept in order to accomplish the aforementioned object is a method of delivering an electric field which delivers the electric field to a region of interest in a 3D subject using an electrode array including a plurality of individual electrodes, comprising: composing an electrode sub-array including a portion of individual electrodes in the electrode array, and sequentially and repeatedly delivering an electric field via at least one pair of the composed electrode sub-arrays.

Here, the electrode sub-array may be capable of varying the intensity of the electric field or the duration of electric field application.

### Advantageous Effects of Invention

According to the inventive concept, the system for delivering an electric field of an embodiment of the inventive concept can independently control voltage or current of the individual electrode by one-to-one connecting a plurality of the generators to the individual electrode via the switching router. In other words, excessive current flow to a specific individual electrode caused by the edge effect of the electrode array can be overcome, and effective thermal management is possible. This can enhance the effectiveness of tumor treatment in the subject.

According to another embodiment of the inventive concept, an electrode sub-array including some of the individual electrodes among a plurality of the individual electrodes contained in the electrode array may be composed. Here, the electrode sub-array may be one individual electrode or include entire individual electrodes. The system for delivering an electric field may sequentially and repeatedly deliver an electric field by connecting a single generator to at least one pair of electrode sub-arrays. Therefore, at least a similar or higher therapeutic effect can be applied to the tumor tissue of the subject compared to conventional techniques, and a lower dose can be delivered to the surrounding tissues to prevent side effects.

### BRIEF DESCRIPTION OF DRAWINGS

FIG. 1 is a drawing illustrating a system for delivering an electric field according to an embodiment of the inventive concept.

FIG. 2 is a schematic view of a system for delivering an electric field.

FIGS. 3 through 6 are the changes in each signal for a switch on and off.

FIG. 3 shows a switch on and off when the switching router and AC generator signals are asynchronous.

FIG. 4 shows a switch on and off when the switching router and generator signals are synchronous.

FIG. 5 shows an embodiment of operating a switch when the switching router and the AC generator signals are synchronized and a signal of the generator is turned off.

FIG. 6 is an embodiment of signal and a drawing for minimizing the leakage current of a FET switch.

FIG. 7 is a schematic view of a daisy-chain temperature measurement system.

FIG. 8 is a schematic circuit diagram of a system for measuring position of a temperature sensor with maximum temperature after measuring the maximum temperature of the electrode.

FIG. 9 shows an embodiment of a system for delivering an electric field in which a switching router is configured using a relay switch of the SPDT type.

FIG. 10 shows a direction of the electric field applied to the object in the embodiment of FIG. 9.

FIGS. 11 to 13 show an embodiment of a system for delivering an electric field in which a switching router includes a switch of the 1P4T type.

FIG. 14 shows a direction of the electric field applied to the object in the embodiment of FIGS. 11 through 13.

FIG. 15 shows an embodiment in which each electrode is individually connected to a generator and a switching router and the electrode is controlled individually.

FIG. 16 is a top view illustrating a direction of the electric field applied to the subject in the embodiment of FIG. 15.

FIG. 17 shows a 3D view illustrating a direction of the electric field applied to the subject in the embodiment of FIG. 15.

FIG. 18 is a block diagram of a sequence of the TTFields using a transducer array layout system.

FIG. 19 is a block diagram of a sequence of the TTFields using a transducer array layout system.

FIG. 20 shows activate and deactivate electrodes in the electrodes attached to the subject in the embodiments of FIGS. 19 and 15.

FIG. 21 is a schematic diagram of connecting one-to-one N generators and N individual electrodes forming each electrode sub-array.

FIG. 22 is a view implementing a schematic diagram of connecting one-to-one N generators and N individual electrodes forming each electrode sub-array.

FIG. 23 is a schematic diagram illustrating an electrode sub-array formed by selecting k electrodes from an electrode array consisting of N individual electrodes.

FIG. 24 shows the electric field-volume histogram delivered to the tumor when individual voltage of an electrode array is optimized for a manikin phantom assuming a hypothetical region of interest (tumor).

FIG. 25 is a schematic diagram of a manikin phantom when a hypothetical region of interest (tumor) is assumed in a manikin phantom and power is applied using a pair of electrode arrays containing a plurality of individual electrodes.

FIG. 26 shows distribution of electric field according to FIG. 25.

FIG. 27 is a schematic diagram of a manikin phantom when a hypothetical tumor is assumed in a manikin phantom and power is applied using a pair of electrode arrays containing a plurality of individual electrodes.

### MODE FOR THE INVENTION

Embodiments of the inventive concept will be described more fully hereinafter with reference to the accompanying drawings and let those skilled in the art implement easily. The inventive concept may be implemented in various forms and not limited to the following some embodiments.

In denoting reference numerals to elements of each drawing, the same reference numerals denote the same elements though the elements are shown in different drawings. In describing embodiments of the inventive concepts, descriptions for universally known elements or functions may be left out when the descriptions make obscure essential points of the inventive concepts.

If there is referred that a part is connected to another part, it includes a case of ‘connecting directly’ with each other by interposing another device as well as a case of ‘connecting electrically’ with each other. In the addition, if there is referred that any part ‘include(s)’ a component, it means that other components are not excluded but further includes another component unless special opposite statement is described.

If there is referred that a part ‘on’ another part, it is on the part or another part may be interposed therebetween. If there is referred that a part ‘directly on’ another part, another part should not be interposed therebetween.

Terms of a first, a second, a third will be used to describe various parts, elements, regions, layers and/or sections, but not limited to these. These terms may be used only to distinguish a part, component, area, layer, or section from another, component, area, layer, or section. Accordingly, the first part, component, area, layer, or section described hereinafter may be referred to as a second part, component, area, layer, or section to the extent that it is not beyond the scope of the inventive concept.

The professional terms will be used hereinafter in order to mention only specific embodiment, but it does not intend to limit the inventive concept. The singular forms used here include plural forms, unless the phrases clearly indicate the opposite meaning. The meaning of “including” in the specification specifies a particular characteristic, domain, integer, step, action, element, and/or component, and does not exclude the presence or addition of other properties, areas, integers, phases, motions, elements, and/or components.

Terms of opposite spaces such as ‘up’ and ‘down’ may be used in order to describe easily a relationship a part with another part illustrated in a drawing. These terms may be intended to include any other meaning or behavior of the device being used, along with the intended meaning in the drawing. For example, when the device on the drawing is turned over, a part described as being ‘under’ other parts are described as being ‘on’ the other parts. Thus, the illustrative term “under” encompasses both of under and on. The device may be rotated 90 degrees or other angles, and terms denoting relative space are interpreted accordingly.

All terms including technical terms and scientific terms used herein, even if not defined differently, have the same meaning as commonly understood by those skilled in the art of the inventive concept. Commonly used dictionary-defined terms are further construed to have meanings consistent with the relevant technical literature and current disclosure, and are not interpreted in an ideal or highly formal sense unless they are defined.

Embodiments of the inventive concept will be described more fully hereinafter with reference to the accompanying drawings and let those skilled in the art implement easily. However, the inventive concept may be implemented in various forms and not limited to the following some embodiments.

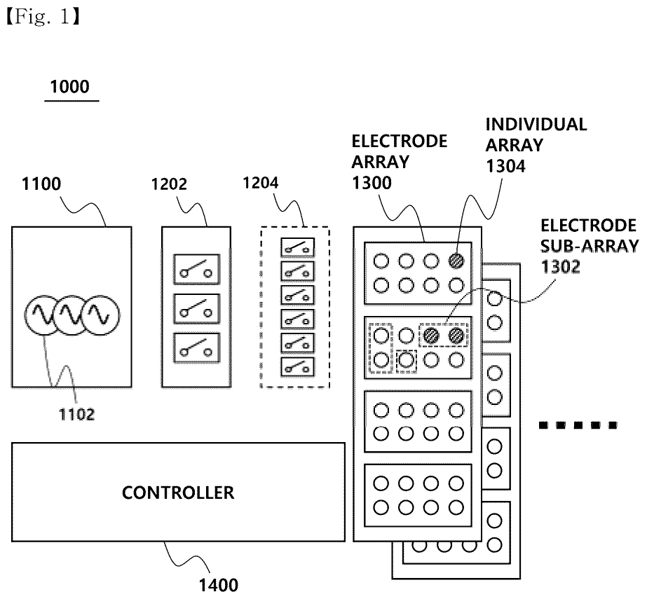

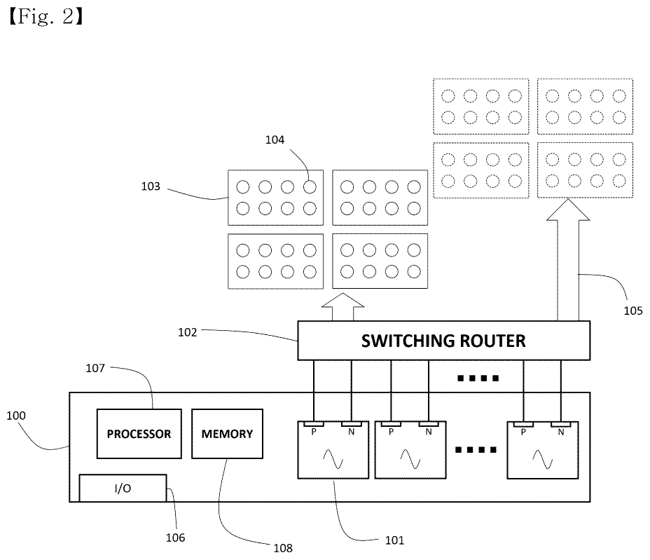

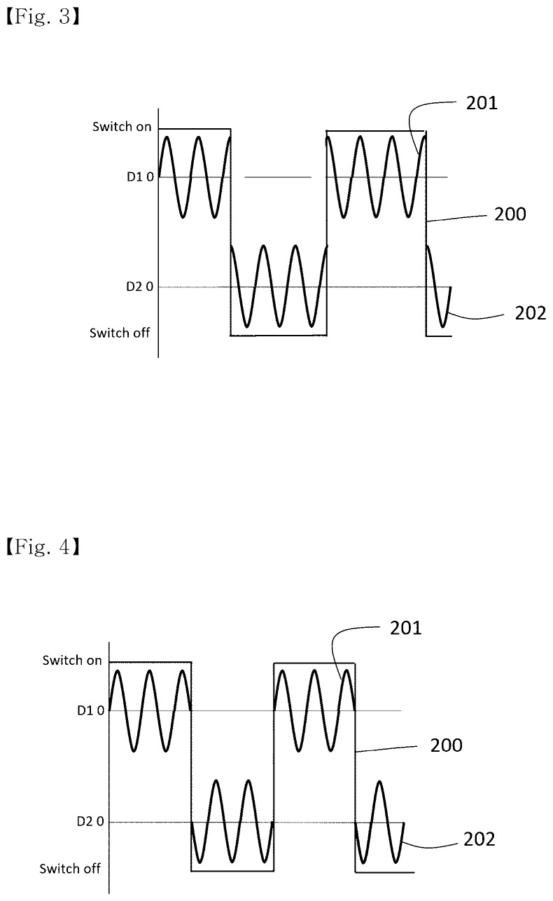

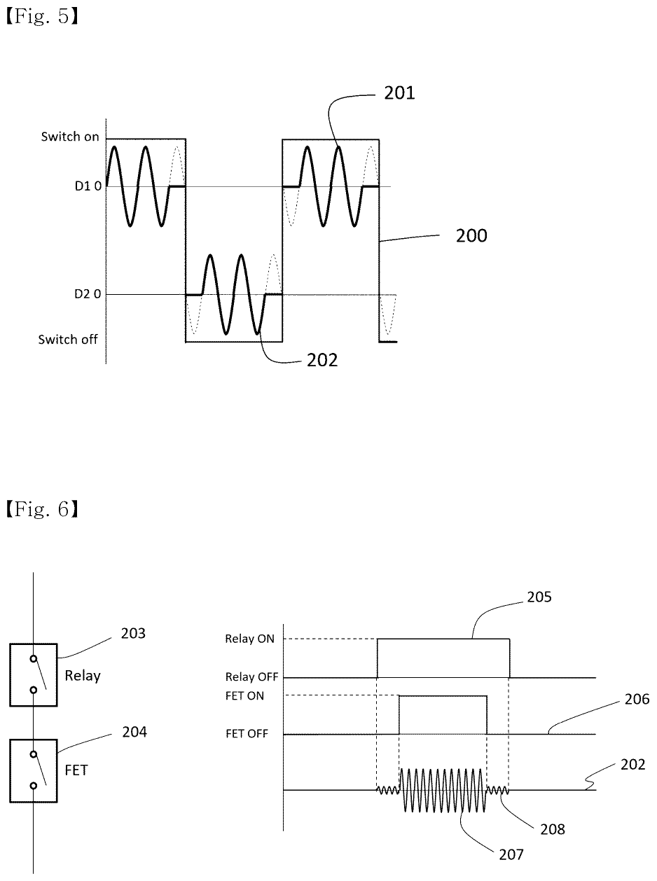

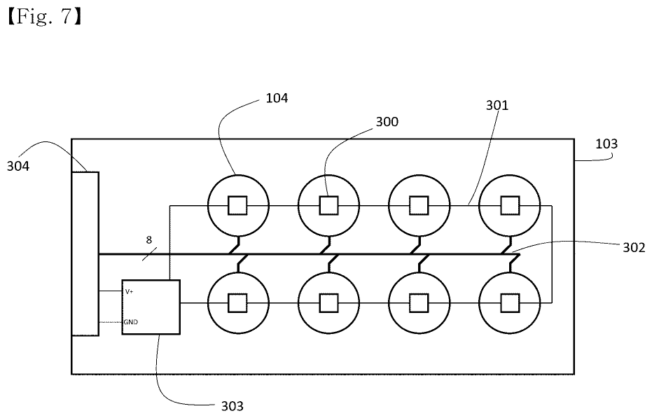

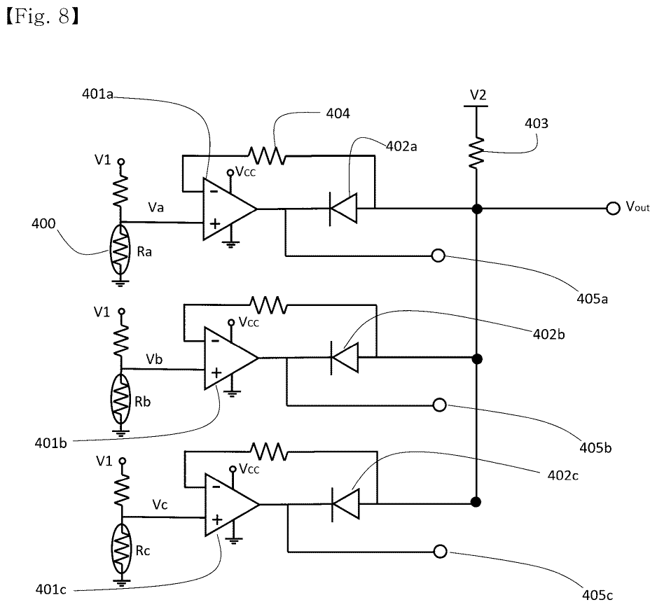

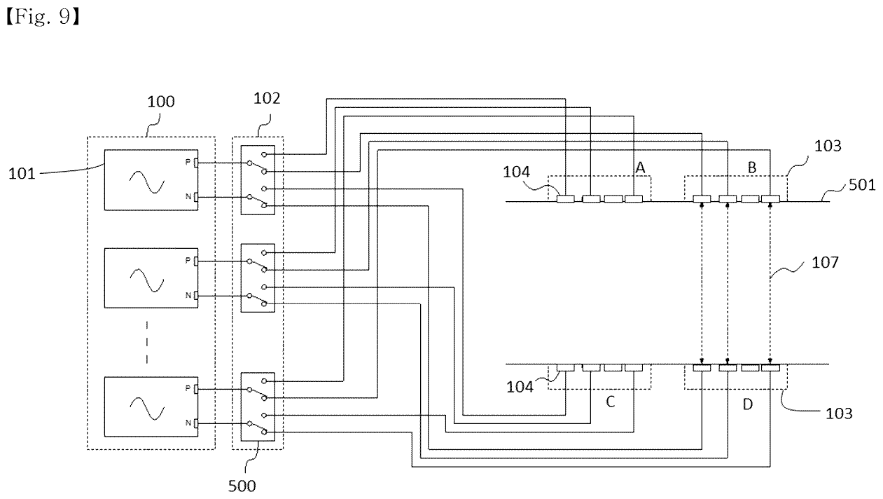

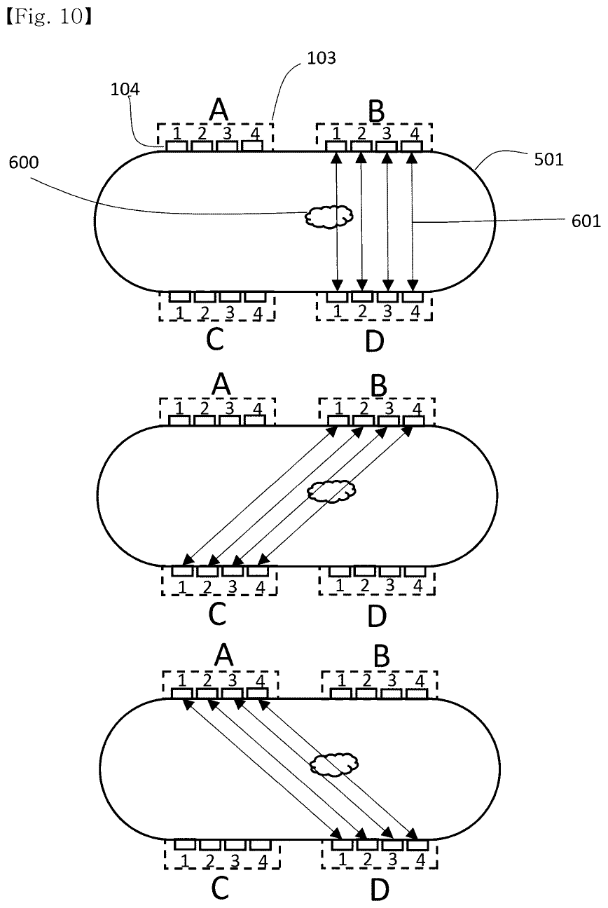

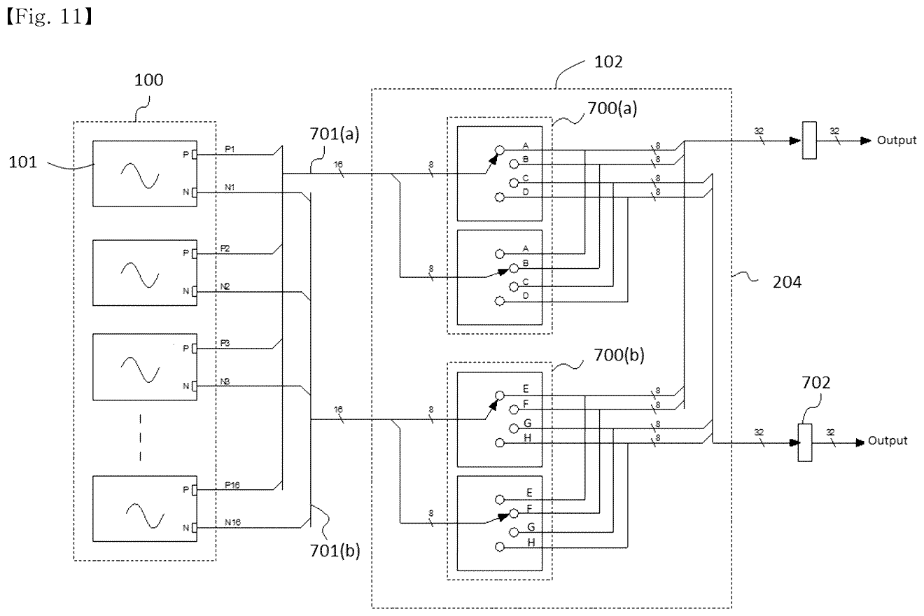

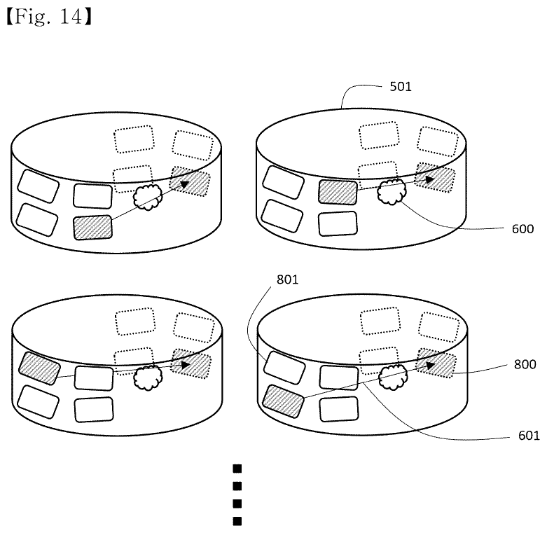

FIG. 1 is a drawing illustrating a system for delivering an electric field according to an embodiment of the inventive concept. FIG. 2 is a schematic view of a system for delivering an electric field. FIGS. 3 through 6 are the changes in each signal for a switch on and off. FIG. 3 shows a switch on and off when the switching router and AC generator signals are asynchronous. FIG. 4 shows a switch on and off when the switching router and generator signals are synchronous. FIG. 5 shows an embodiment of operating a switch when the switching router and the AC generator signals are synchronized and a signal of the generator is turned off. FIG. 6 is an embodiment of signal and a drawing for minimizing the leakage current of a FET switch. FIG. 7 is a schematic view of a daisy-chain temperature measurement system. FIG. 8 is a schematic circuit diagram of a system for measuring position of a temperature sensor with maximum temperature after measuring the maximum temperature of the electrode. FIG. 9 shows an embodiment of a system for delivering an electric field in which a switching router is configured using a relay switch of the SPDT type. FIG. 10 shows a direction of the electric field applied to the object in the embodiment of FIG. 9. FIGS. 11 to 13 show an embodiment of a system for delivering an electric field in which a switching router includes a switch of the 1P4T type. FIG. 14 shows a direction of the electric field applied to the object in the embodiment of FIGS. 11 through 13.

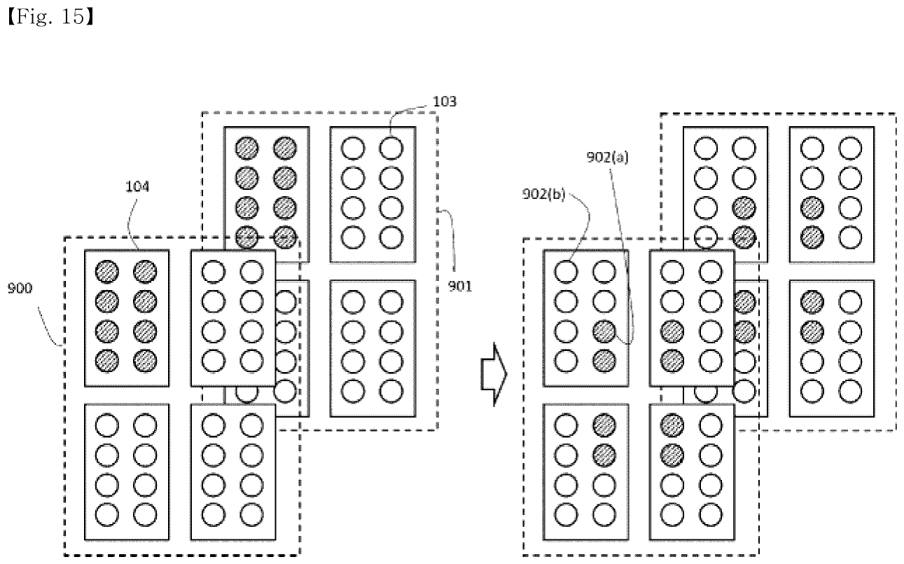

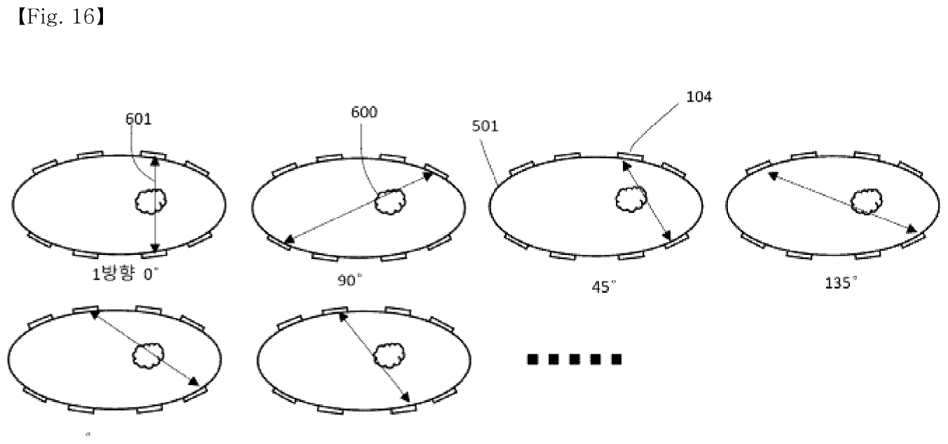

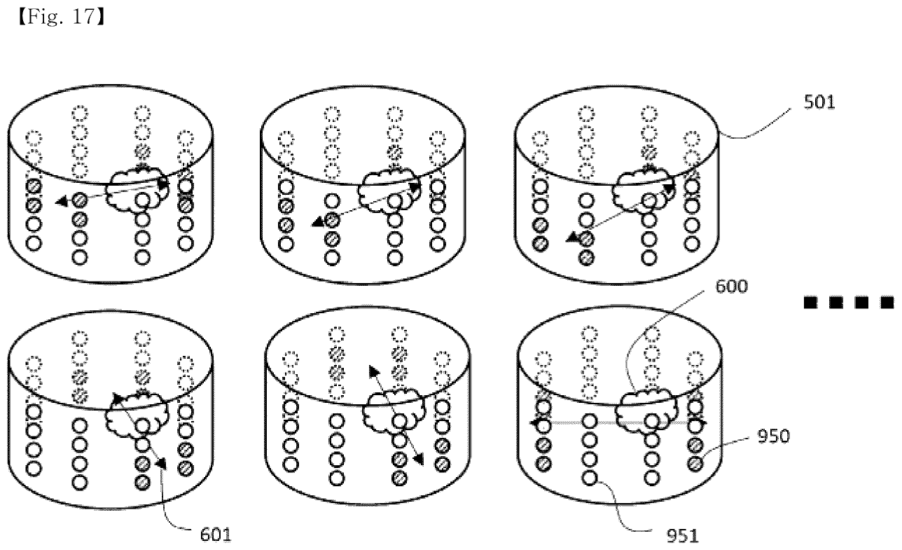

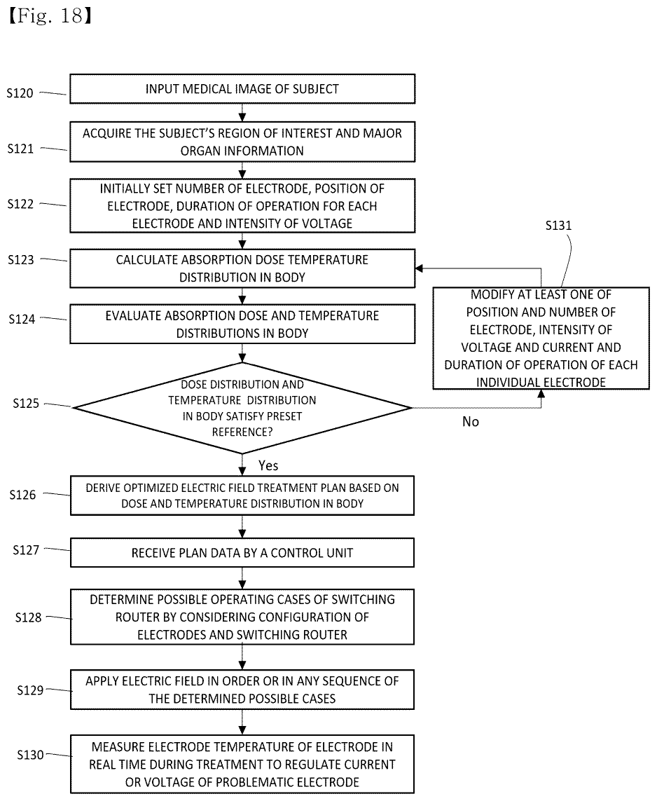

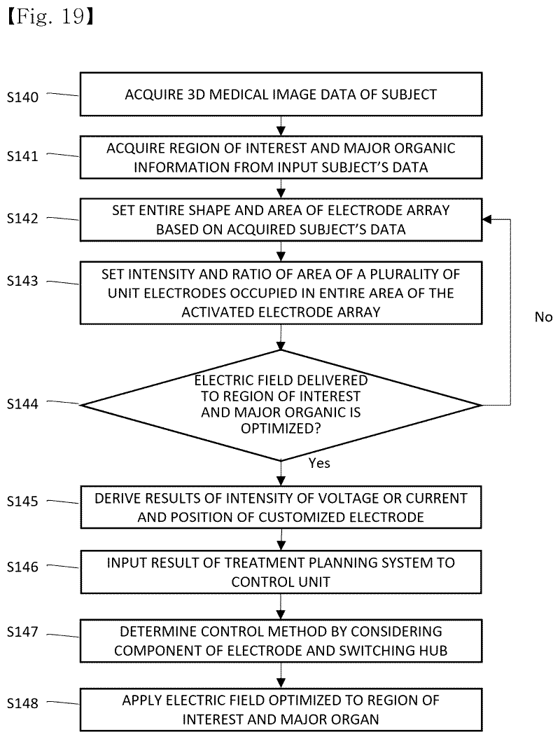

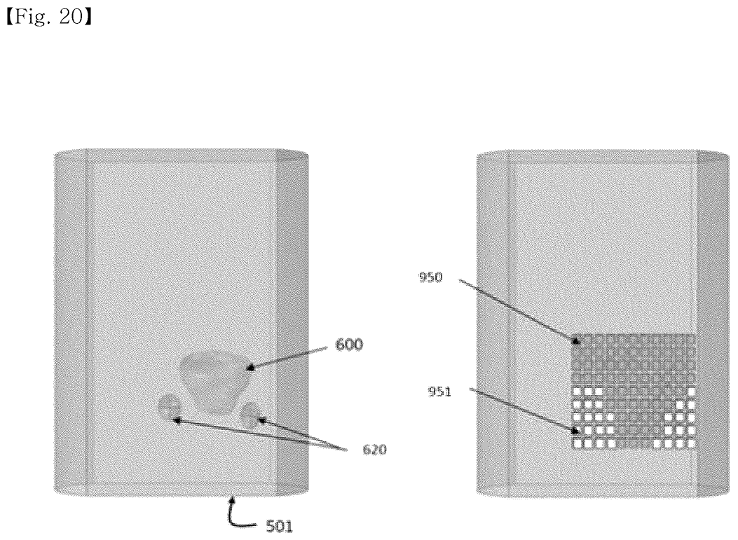

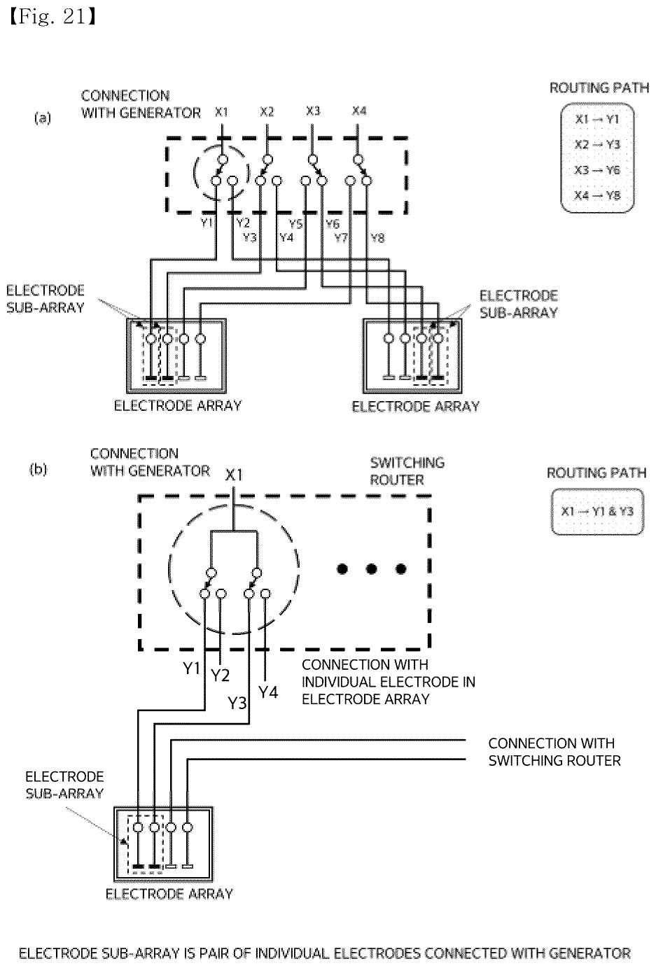

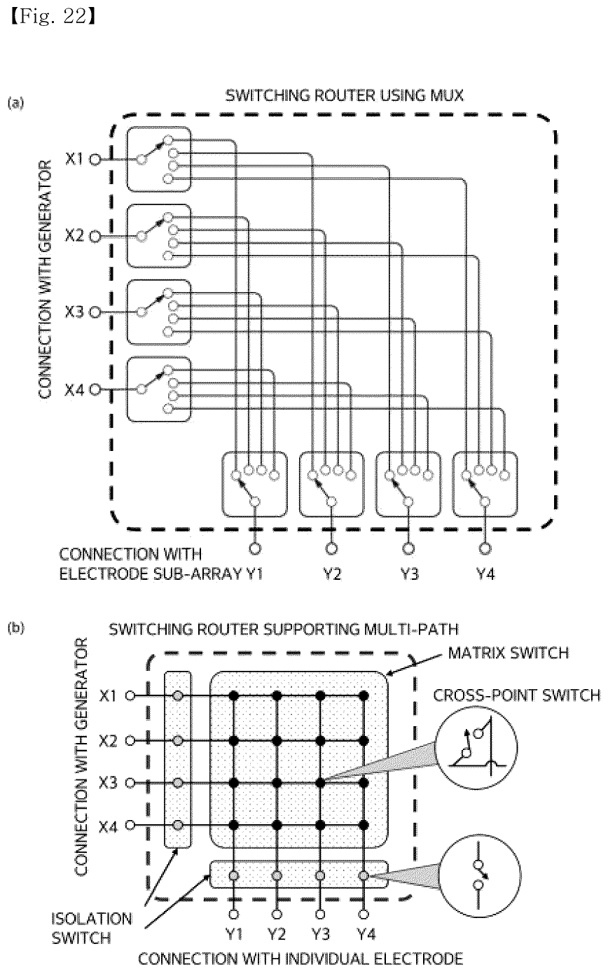

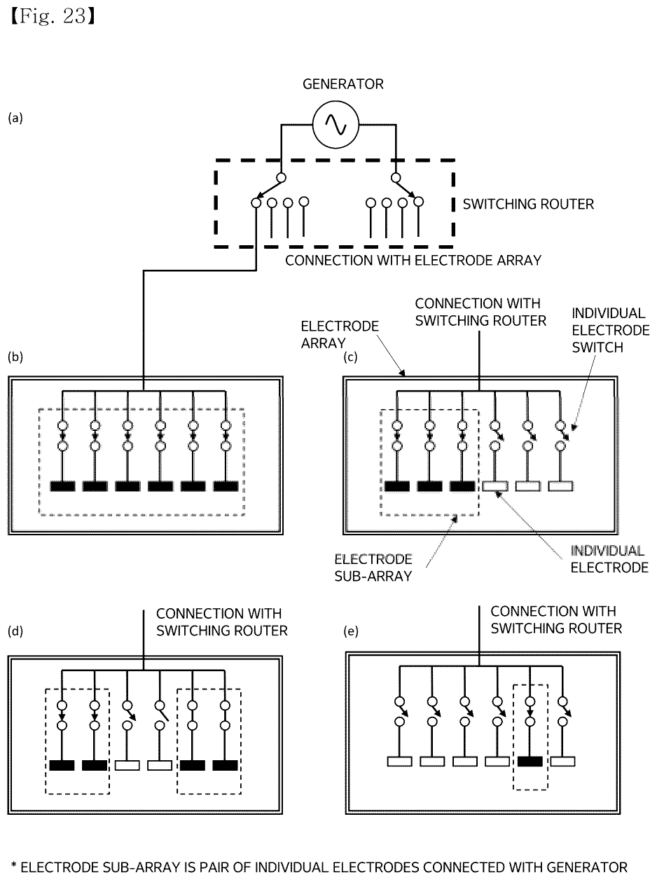

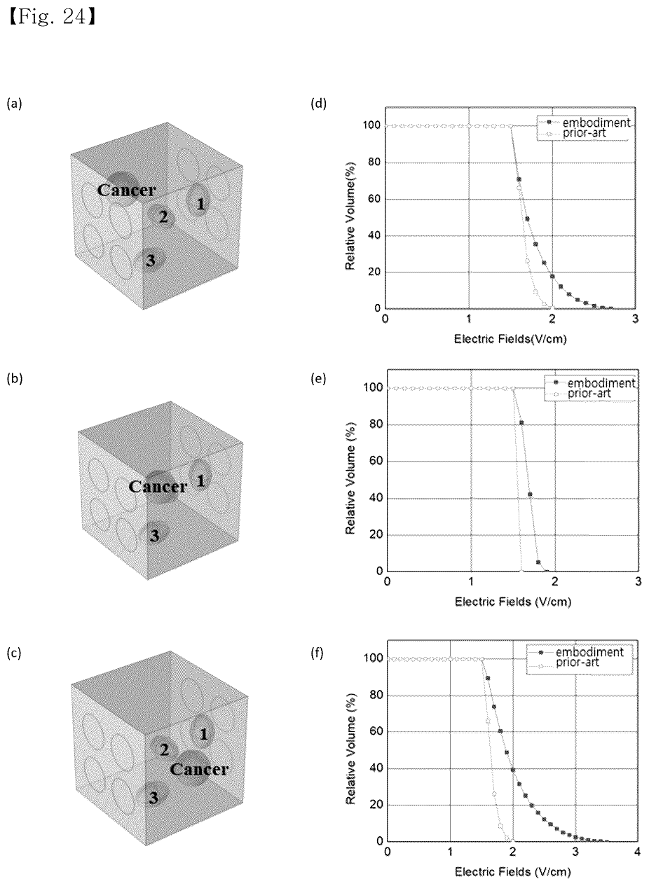

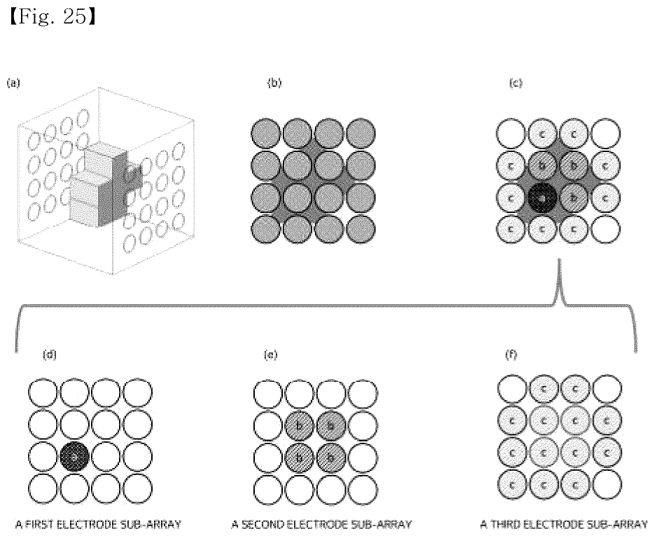

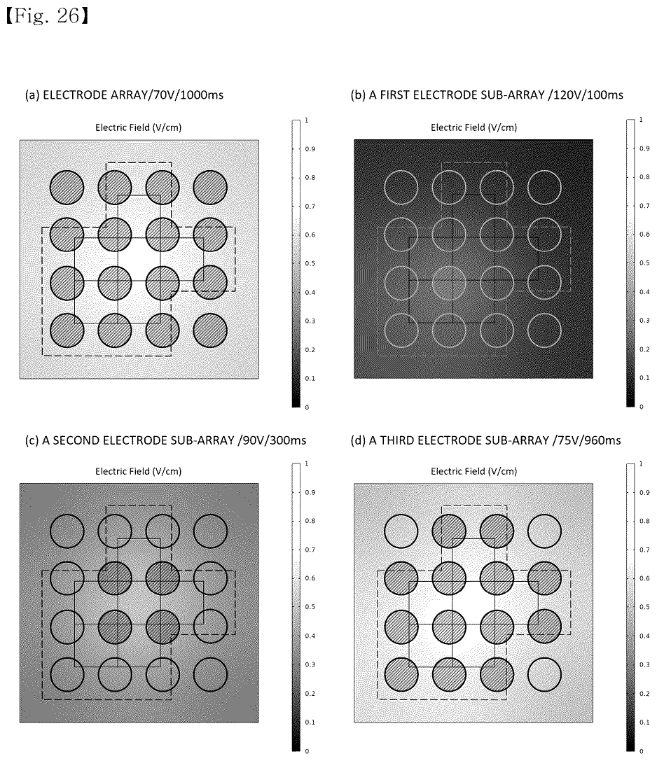

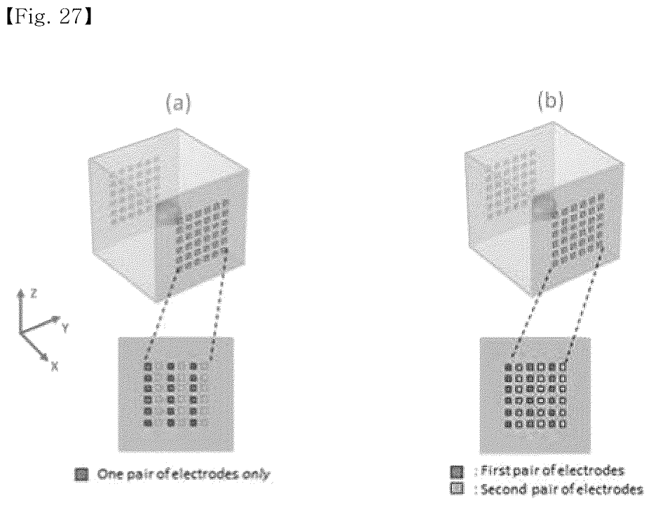

FIG. 15 shows an embodiment in which each electrode is individually connected to a generator and a switching router and the electrode is controlled individually. FIG. 16 is a top view illustrating a direction of the electric field applied to the subject in the embodiment of FIG. 15. FIG. 17 shows a 3D view illustrating a direction of the electric field applied to the subject in the embodiment of FIG. 15. FIG. 18 is a block diagram of a sequence of the TTFields using a transducer array layout system. FIG. 19 is a block diagram of a sequence of the TTFields using a transducer array layout system. FIG. 20 shows activate and deactivate electrodes in the electrodes attached to the subject in the embodiments of FIGS. 19 and 15. FIG. 21 is a schematic diagram of connecting one-to-one N generators and N individual electrodes forming each electrode sub-array. FIG. 22 is a view implementing a schematic diagram of connecting one-to-one N generators and N individual electrodes forming each electrode sub-array. FIG. 23 is a schematic diagram illustrating an electrode sub-array formed by selecting k electrodes from an electrode array consisting of N individual electrodes. FIG. 24 shows the electric field-volume histogram delivered to the tumor when individual voltage of an electrode array is optimized for a manikin phantom assuming a hypothetical region of interest (tumor). FIG. 25 is a schematic diagram of a manikin phantom when a hypothetical region of interest (tumor) is assumed in a manikin phantom and power is applied using a pair of electrode arrays containing a plurality of individual electrodes. FIG. 26 shows distribution of electric field according to FIG. 25. FIG. 27 is a schematic diagram of a manikin phantom when a hypothetical tumor is assumed in a manikin phantom and power is applied using a pair of electrode arrays containing a plurality of individual electrodes.

Referring to FIG. 24, FIG. 24 shows the electric field-volume histogram delivered to the tumor when individual voltage of an electrode array is optimized for a manikin phantom assuming a hypothetical region of interest (tumor). In FIG. 24(a) to 24(c), the tumor was positioned differently, and in each case, the voltage of each individual electrode in the electrode array was optimized by a transducer array layout system.

The electric field-volume histogram simulated the electric field according to the conventional method (same voltage) and the optimization method, and presented the results of the analysis in a manner similar to the dose-volume histogram, which is a tool for evaluating radiotherapy planning. These are shown in FIG. 24(d) to 24(f), with the horizontal axis representing intensity of the electric field and the vertical axis representing the proportion of exceeding the intensity of the electric field intensity of the horizontal axis in the total volume of tumor tissue. In FIG. 24(d) to 24(f), the square symbol results in the present embodiment, and the circle symbol is in accordance with the conventional art. As shown in FIG. 24(d) to 24(f), it can be seen that the optimization method delivers more electric field to the tumor tissue than the conventional art. However, since leakage current through the skin is unavoidable if the voltages of the individual electrodes are different, a special method in which the individual electrodes of the electrode array are divided into electrode sub-arrays forming an equipotential surface to connect sequentially with the generators of the system for delivering an electric field must be considered in order to implement the optimization method.

Referring to FIGS. 1 to 27, a system for delivering an electric field according to an embodiment of the inventive concept is a system for delivering an electric field 1000 that delivers an electric field to a region of interest in a 3D subject using an electrode array having a plurality of individual electrodes, the system comprising: a plurality of generators 1100 providing the electric field; a plurality of electrode sub-arrays 1300 delivering alternating current power, generated by the plurality of generators, to the subject, each electrode sub-array 1300 including a portion of the individual electrodes in the electrode array; a switching router 1202 one-to-one connecting the plurality of generators 1100 and the plurality of electrode sub-arrays 1300; and a controller 1400 controlling the plurality of generators 1100, the plurality of electrode sub-array 1300, and the switching router 1202.

The plurality of generators 1100 is configured to generate alternating current power in order to deliver an electric field to the region of interest in the three-dimensional subject. The plurality of generators is configured to control the magnitude of voltage or current to be delivered to the region of interest in the three-dimensional subject.

The plurality of electrode sub-arrays 1302 and at least one individual electrode 1304 is configured to deliver the alternating current power generated from the plurality of generators 1100 to the subject, and each of the plurality of electrode sub-arrays 1302 may include a part of the plurality of the individual electrodes 1304 in the electrode array 1300.

That is, the electrode sub-array 1302 may include one, two, three and four or more of the individual electrodes 1304 in the electrode array 1300, and may include a plurality of individual electrodes included in at least one row of the plurality of individual electrodes 1304 in the electrode array 1300, or may include a plurality of individual electrodes in at least one column.

The switching router 1202 is configured to connect the plurality of generators 1100 and the plurality of electrode sub-arrays 1302 in a one-to-one manner.

The switching router 1202 may be included in order to connect the plurality of generators 1100 and the plurality of electrode sub-arrays 1302 in a one-to-one manner. Thus, the plurality of generators 1102 and the plurality of electrode sub-arrays 1302 may be connected or disconnected.

The controller 1400 is configured to control the plurality of generators 1100, the plurality of electrode sub-arrays 1302 and the switching router 1202. The controller 1400 is configured to control the switching router 202 to provide connection in order to provide electric field delivered from each of the plurality of the generators 1100 to the plurality of the electrode sub-arrays.

The controller 1400 is configured to provide an independently adjustable current to each of the plurality of electrode sub-arrays to prevent excessive current from being applied to a particular individual electrode.

Referring to FIGS. 21 and 22 together, each of N electrode sub-arrays including at least one individual electrode and N generators may be 1:1 connected to apply independent current and voltage to each electrode sub-array, thereby preventing excessive current from flowing to each electrode sub-array. In addition, it may be also possible to control each electrode sub-array in a manner as the first duration to the first electrode sub-array and the second duration to the second electrode sub-array, and to repeat this control sequentially.

In particular, an electrode sub-array may be formed of one individual electrode, and in this case, a generator may be connected to each individual electrode. In addition, it may be possible that two individual electrodes in the same electrode array are connected to different generators.

Specifically considering, FIG. 21(a) shows a 1:1 connection between generators (X1, X2, etc.) and electrode sub-arrays (Y1, Y2, etc.), in particular, when an electrode sub-array is formed of one individual electrode. FIG. 22(a) shows that inputs and outputs of the switching router may be composed of a plurality of MUX in order to implement a connection such as FIG. 21(a). In other words, as shown in FIG. 22(a), a generator connected to X1 may be connected 1:1 to an electrode sub-array formed by one individual electrode of Y1.

FIG. 21(b) shows a 1:1 connection between a generator and an electrode sub-array, but in a different type, an electrode sub-array formed by two individual electrodes. FIG. 22(b) shows a switching router that supports multiple paths to implement a connection such as FIG. 21(b). In order to connect N generator inputs and M individual electrodes, each node of a matrix switch consisting of N rows and M columns may be composed of a cross-point switch, thereby allowing multiple Y outputs for a single X input, and an isolation switch may be turned on and off for each node of input and output. Naturally, a single output should not be connected to a plurality of inputs. In other words, in order to implement a connection such as FIG. 21(a), a generator connected to X1 as shown in FIG. 22(b) may be connected to multiple paths of Y1 and Y3.

On the other hand, the controller 1400 may independently control a magnitude of voltage or current for each of the plurality of generators 1100, and independently control duty cycle of applying voltage or current for each of the plurality of generators 1100. In other words, the controller 1400 is configured to control each of the plurality of the generators 1100 to adjust dose of electric field delivered to the subject, by a method of controlling the intensity of voltage or current for each of the generators, or controlling the duty cycle of applying voltage or current for each of the generators.

In addition, the controller 1400 is configured to determine a mapping method between the plurality of generators 1100 and the plurality of electrode sub-arrays 1302, and control the switching router 1202 in accordance with the mapping method. In other words, the control unit 1400 is configured to determine the mapping method between the plurality of generators 1100 and the plurality of electrode sub-arrays 1302, and control a connection between the plurality of generators 1100 and the plurality of electrode sub-arrays 1302 in accordance with the determined mapping method. In order to control the connection, the controller 1400 is configured to control the switching router 1202.

Here, the switching router 1202 is configured to connect the plurality of generators 1100 and the plurality of electrode sub-arrays 1302 to be mapped. In other words, the switching router 1202 is configured to control the connection between the plurality of generators and the plurality of electrode sub-arrays according to the mapping method.

In particular, the switching router 1202 may include a relay switch, and the switching router 1202 may further include a field-effect transistor FET switch that is connected in series with the relay switch.

The relay switch may have problems with a chattering phenomenon and slow response times during operation, and field-effect transistors may have problems with leakage current in an off state. However, when the switching router 1202 is configured to connect the relay switch and the field-effect transistor switch in series, a fast response time and minimized leakage current may be obtained if the field-effect transistor switch is operated within a short period of time to apply current and voltage after the relay switch is activated. In addition, even when the switching router is turned off, there is an effect of minimizing the response time and leakage current if the field-effect transistor switch is turned off first and the relay switch is turned off.

In addition, each individual electrode may further include a temperature sensor (not shown) that detects a temperature rise, and the controller 1400 is configured to apply current to each electrode sub-array 1302 where a temperature rise is detected by the temperature sensor by independently reducing duty cycle of applying current, or apply voltage and current of the generator connected to each electrode sub-array to each electrode sub-array 1302 where a temperature rise is detected by the temperature sensor by independently decreasing intensity of voltage and current.

In other words, if the temperature of each individual electrode rises beyond the predetermined temperature, damage may occur to the subject, thus it will be necessary to reduce the electric field delivered to the electrode sub-array 1302 in which the temperature rise is detected in the temperature sensor in order to minimize the damage to the subject. Accordingly, the electric field delivered to the subject may be reduced using a method of applying current to the electrode sub-array 1302 where a temperature rise is detected in the temperature sensor by reducing duty cycle of applying current, or applying voltage and current of the generator connected to the electrode sub-array to the electrode sub-array 1302 where a temperature rise is detected in the temperature sensor by decreasing intensity of voltage and current.

Here, the controller 1400 may receive electrode layout information from an external treatment planning system or an external transducer array layout system, and the electrode layout information may include the position of the electrode array, the selection of the electrode sub-array, and the voltage or current intensity of the electrode sub-array. In particular, the position of the electrode array, including the plurality of individual electrodes, may be represented in a 3D model of the subject.

In addition, in accordance with the provided electrode layout information, the controller 1400 may determine the mapping method between the plurality of generators and the plurality of electrode sub-arrays, and is configured to control the switching router 1202 according to the mapping method, and the controller 1400 may set a connection order and a duration of operation of the electrode sub-array, and determine the mapping method between the active and inactive electrode sub-arrays and the plurality of generators for each mode of operation and the initial value of voltage or current of each generator, according to the provided electrode layout information.

Referring to FIGS. 1 to 27 again, a system for delivering an electric field according to an embodiment of the inventive concept is a system for delivering an electric field which delivers the electric field to a region of interest in a 3D subject using an electrode array having a plurality of individual electrodes, including: a generator 1102 providing the electric field; an electrode sub-array 1300 delivering alternating current power, generated by the generator 1102, to the subject, the electrode sub-array 1302 including a part of the plurality of individual electrodes in the electrode array 1300; a switching router 1202 connecting the generators 1102 and the electrode sub-array 1302; at least one electrode switch 1204 connecting the electrode sub-array 1302 and at least one of the individual electrode 1304; and a controller 1400 controlling the plurality of generators 1102, the plurality of electrode sub-array 1302, the switching router 1202 and the electrode switch 1204.

The generator 1102 is configured to generate alternating current power and control the magnitude of voltage or current delivered to the region of interest in the three-dimensional subject.

The electrode sub-array 1302 is configured to deliver the alternating current power generated from the generator 1102 to the subject, and the electrode sub-array 1302 may include a part of the plurality of individual electrodes 1304 in the electrode array 1300. In other words, the electrode sub-array may include one, two, three and four or more of the plurality of individual electrodes 1304 in the electrode array 1300, and include a plurality of individual electrodes in at least one row of the plurality of individual electrodes 1304 in the electrode array 1300 or a plurality of individual electrodes in at least one column.

The switching router 1202 connects or disconnects the generator 1102 and the electrode sub-array 1302 under control of the controller.

At least one of the electrode switches 1204 connects or disconnects the electrode sub-array 1302 and at least one individual electrode 1304 under the control of the controller.

The controller 1400 is configured to control the generator 1102, the electrode sub-array 1302 and the switching router 1202. In other words, the controller may control the switching router 1202 to control the connection between the generator 1102 and the electrode sub-array 1302, control the electrode switch 1204 to control the connection between the electrode sub-array 1302 and at least one individual electrode 1304, and control alternating current power generated by the generator 1102.

Here, the controller 1400 is configured to select at least one electrode sub-array and provide adjustable current to ensure that the dose distribution matches the location, shape, and size of the region of interest.

Referring to FIG. 23, the controller 1400 is configured to select k individual electrodes from an electrode array consisting of N individual electrodes to compose the electrode sub-array, where, k may be 1, 2, 3, . . . , N, and a first voltage may be applied to a first electrode sub-array for a first duration, or a second voltage may be applied to a second electrode sub-array for a second duration. By repeating this control sequentially, it will be possible to deliver the dose of the electric field optimized for the shape of the region of interest.

In particular, the electrode sub-array is configured to select entire the individual electrodes in the electrode array as the electrode sub-array as shown in FIG. 23(b), three individual electrodes from six individual electrodes in the electrode array may be selected as the electrode sub-array and as shown in FIG. 22(c), four individual electrodes from the six individual electrodes in the electrode array may be selected as the electrode sub-array as shown in FIG. 23(d), it is possible to select only one individual electrode from the six individual electrodes in the electrode array as the electrode sub-array as shown in FIG. 23(e).

On the other hand, the controller 1400 is configured to control a magnitude of voltage or current for the generator 1102, and control duty cycle of applying voltage or current for the generator 1102. In other words, the control unit 1400 may control the generator 1102 to adjust dose of electric field delivered to the subject by a method of controlling the intensity of voltage or current for each of the generators, or controlling the duty cycle of applying voltage or current for each of the generators.

The switching router 1202 and the electrode switch 1204 may include a relay switch, and the switching router 1202 and the electrode switch 1204 may further include a field-effect transistor switch that is connected in series with the relay switch.

For example, when the switching router 1202 and electrode switch 1204 are configured to connect the relay switch and the field-effect transistor switch in series, leakage current may occur when the FET switch is off, but if the field-effect transistor switch is operated within a short time after the relay switch is activated to apply the current and voltage, the fast response time may be ensured and leakage current may be minimized. In addition, even when switching router 1202 and an electrode switch 1204 are turned off, the response time and leakage current may be minimized if the field-effect transistor switch is turned off and then the relay switch is turned off.

In addition, each individual electrode in the electrode sub-array 1302 may further include a temperature sensor (not shown) that detects a temperature rise, and the controller 140 is configured to apply current to each electrode sub-array 1302 where a temperature rise is detected by the temperature sensor by reducing duty cycle of current application, or apply voltage and current of the generator connected to each electrode sub-array to each electrode sub-array 1302 where a temperature rise is detected by the temperature sensor by decreasing intensity of voltage and current.

In other words, if the temperature of each individual electrode rises beyond the predetermined temperature, damage may occur to the subject, thus it will be necessary to reduce the electric field delivered to the electrode sub-array 1302 in which the temperature rise is detected in the temperature sensor in order to minimize the damage to the subject. Accordingly, the electric field delivered to the subject may be reduced using a method of applying current to the electrode sub-array 1302 where a temperature rise is detected in the temperature sensor by reducing duty cycle of applying current or applying voltage and current of the generator connected to the electrode sub-array to the electrode sub-array 1302 where a temperature rise is detected in the temperature sensor by decreasing intensity of voltage and current.

On the other hand, the electrode sub-array 1302 may include at least one individual electrode and may include individual electrodes forming a row of the electrode array, or individual electrodes forming a column of the electrode array.

Further, the controller may receive electrode operation information comprising i) a connection selection or not, ii) a duration of operation and iii) an operation power for the electrode sub-array and a plurality of external electrode sub-array from an external transducer array layout system to implement sequentially and repeatedly the connection selection and applying operation power for the duration of operation for the electrode sub-array and a plurality of external electrode sub-array.

For example, the controller 140 may receive electrode operation information comprising i) a connection selection or not, ii) a duration of operation and iii) an operation power for the electrode sub-array and a plurality of external electrode sub-array from an external transducer array layout system to establish an operation plan, and implement sequentially and repeatedly the connection selection and applying operation power for the duration of operation for the electrode sub-array and a plurality of external electrode sub-array according to the operation plan. In other words, each electrode sub-array may be connected sequentially, and the operating power may be applied repeatedly according to the duration of operation.

A method of delivering an electric field according to an embodiment of the inventive concept in order to accomplish the aforementioned object is a method of delivering an electric field which delivers the electric field to a region of interest in a 3D subject using an electrode array having a plurality of individual electrodes, including: forming an electrode sub-array including a part of individual electrodes in the electrode array, and sequentially and repeatedly delivering an electric field via at least one or more pairs of the constructed electrode sub-arrays.

In particular, intensity of the electric field or duration of applying the electric field may be different from each of the electrode sub-arrays.

FIG. 24 shows the electric field-volume histogram delivered to the tumor when individual voltage of an electrode array is optimized for a manikin phantom assuming a hypothetical region of interest (tumor). In FIG. 24(a) to 24(c), the tumor was positioned differently, and in each case, the voltage of each individual electrode in the electrode array was optimized by a transducer array layout system.

The electric field-volume histogram simulated the electric field according to the conventional method (same voltage) and the optimization method, and presented the results of the analysis in a manner similar to the dose-volume histogram which is a tool for evaluating radiotherapy planning. These are shown in FIG. 24(d) to 24(f), with the horizontal axis representing intensity of the electric field and the vertical axis representing the proportion of the total volume of tumor tissue exceeding the intensity of the electric field intensity of the horizontal axis. In FIG. 24(d) to 24(f), the square symbol represents the present embodiment, and the circle symbol represents the conventional art. As shown in FIG. 24(d) to 24(f), it can be seen that the optimization method delivers more electric field to the tumor tissue than the conventional art. However, since leakage current through the skin is unavoidable if the voltages of the individual electrodes are different, a special method in which the individual electrodes are divided into electrode sub-arrays forming an equipotential surface to connect sequentially with the generators of the system for delivering an electric field must be considered in order to implement the optimization method.

FIG. 25 is a schematic diagram of a manikin phantom when a hypothetical region of interest (tumor) is assumed in a manikin phantom and power is applied using a pair of electrode arrays containing a plurality of individual electrodes. FIG. 25(a) shows a hypothetical tumor, and it is supposed that a pair of electrode arrays as shown in the figure. FIG. 25(b) shows an array of electrodes on one side of them. From the point of view of this electrode array, the shape of the tumor is shown on the individual electrodes. In the conventional method, a generator is connected to this electrode array and delivers an electric field to the subject phantom. The individual electrodes all form the same equipotential surface. An electric field may be delivered through the equipotential surface throughout the tumor, but there may be also delivered around the tumor. However, it is advantageous to deliver the maximum dose to the tumor volume for therapeutic effect but the minimum dose outside the tumor volume to prevent side effects. Here, the dose may be the unit of energy that take time into account, i.e., the absorbed energy or the absorbed energy density.

For this purpose, it may be determined by optimization which voltage should be applied to each individual electrode or the electrode sub-array through an external transducer array layout system. At this time, it may be considered that the maximum current is determined according to the specifications of the generator. FIG. 25(c) shows the voltage of each individual electrode determined above. The determination of the voltage that can be applied to each electrode sub-array must consider the current and voltage specifications of the generator and the input impedance experienced by each individual electrode or electrode sub-array. For example, if an electrode sub-array consisting of 10 individual electrodes with a diameter of 1.8 cm has an input impedance of 150Ω and the generator has a maximum specification of 1 A and 100 V, the current is limited to 667 mA. If the input impedance of the electrode sub-array is 50Ω, the voltage is limited to 50 V. In addition, a predetermined maximum current density may be used to determine the voltage that can be applied to each electrode sub-array. The approval time of each electrode sub-array may be optimized through linear programming, which considers conditions to maximize the dose delivered to the tumor tissue and minimize the dose delivered to the surrounding tissue.

In this embodiment, using the electrode sub-array and voltage information determined in the external transducer array layout system, it is divided into three operation modes as shown in FIG. 25(d) to 25(f). For example, the electrode switch of each individual electrode may be controlled to connect four individual electrodes and the generator such that a second electrode sub-array is formed as the configuration of FIG. 25(e). In this manner, three operation modes may be sequentially and cyclically performed, in which a first electrode sub-array may be connected and a first voltage may be applied for a first duration, a second voltage may be applied to a second electrode sub-array for a second duration, and a third voltage may be applied to a third electrode sub-array for a third duration.

FIG. 26 shows an electric field distribution according to FIG. 25. In FIG. 26(a) to 26(d), the inner solid line represents a tumor tissue, and the inner dotted line represents a surrounding tissue. FIG. 26(a) shows the electric field distribution in a cross-section of the tumor when 70V is applied for 1,000 ms using an electrode array by the conventional method. FIG. 26(b) to 26(d) show the electric field distribution in the same cross-section when 100V is applied to a first electrode sub-array for 100 ms, 90V is applied to a second electrode sub-array for 300 ms and 75V is applied to a third electrode sub-array for 960 ms.

On the other hand, the electrode sub-array 1302 may include at least one individual electrode and may include individual electrodes forming a row of the electrode array, or individual electrodes forming a column of the electrode array.

FIG. 27 is a schematic diagram of a manikin phantom when a hypothetical tumor is assumed in a manikin phantom and power is applied using a pair of electrode arrays containing a plurality of individual electrodes. Referring to FIG. 27, an electrode sub-array which is formed by columns or rows may be composed from the electrode array. In other words, a first electrode sub-array may be formed of individual electrodes in a column as shown in FIG. 27(a), the first electrode sub-array may be formed of individual electrodes in a first pair of electrodes, and a second electrode sub-array may be formed of individual electrodes in a second pair of electrodes, as shown in FIG. 27(b). The operation mode, in which a first electric field is applied to the first electrode sub-array for a first duration and a second electric field is applied to the second electrode sub-array for the second duration, may be repeated cyclically to deliver an electric field to the subject.

On the other hand, the input impedance experienced by the individual electrodes composing the electrode array that delivers the tumor treating fields changes in real time due to factors such as the edge effect depending on the structure and shape of the electrode array, the change in properties of the hydrogel between the individual electrode and the skin, the change in impedance of the skin, and the change in the long-term or circadian rhythm of the impedance in the body. In addition, the change in contact impedance due to the replacement of the electrode array is also an important factor.

In particular, if the intensity of current flowing to a particular individual electrode increases by more than a certain intensity depending on the edge effect and the arrangement of the electrode arrays, the temperature rises, and the risk of skin burns and a decrease in the therapeutic effect are inevitably occurred. In a conventional method, if the temperature of the individual electrodes rises above the limit temperature (41 degrees Celsius), the treatment is discontinued until the temperature is below the limit temperature again. This means that the time when the actual electric field is delivered to the target area may be less than the half of the total time of attaching the electrode array to the body and activating the treatment device, and thus this is a factor that significantly reduces the effectiveness of the treatment.

A first method that can be considered as a way to solve this problem is a method of reducing the overall current if the temperature of the individual electrodes is above the limit temperature. However, the reduction in the total current reduces the intensity of the electric field delivered to the tumor and thus reduces the effectiveness of the treatment.

Second, it is possible to connect an electrode switch to each individual electrode in series and control the on/off of current flowing to each electrode based on the temperature measurement of each electrode. It is expressed as enable/disable the switching of the current at each electrode.

For example, when a current of 900 mA is applied to an electrode array including nine individual electrodes connected in parallel, if the temperature rise of a particular electrode is detected and it is determined that the current of the particular electrode is controlled by a 90% duty cycle, the total average current is 890 mA (8×100 mA+1×0.9×100 mA), and only a current reduction of 1.1% can be used to slow or prevent the temperature rise of a particular electrode. In addition, if the current of the generator (AC signal generator) can be increased by 1%, the overall average current is 898.9 mA (8×101 mA+1×0.9×101 mA), which means that only a 0.1% decrease in current for 900 mA is enough to prevent the temperature rise of a particular electrode. However, considering that a plurality of individual electrodes in a single electrode array are connected in parallel to a current-path lead line out of the generator, a separate means of controlling the magnitude of the total current according to the opening and closing of a specific electrode may be required in order to implement the above means. In addition, the opening and closing of the electrode causes a sudden change in the load impedance experienced by the generator, so the occurrence of spikes is inevitable and a separate means are required to eliminate them.

The effect of the tumor treating fields is related to the angle between the direction of the electric field and the direction of the axis of division of the tumor or cancer cell. When the two directions coincide, the division is greatly hindered, and when the two directions intersect at right angles, the division is almost unhindered. Therefore, the conventional technique applies the electric fields in the direction in which the directions of electric fields are orthogonal. Nonetheless, it is inevitable that there is an invalidity angle that does not receive the therapeutic effect of the tumor treating fields. The inventive concept is intended to overcome the difficulties of this technique.

According to the inventive concept, the system for delivering an electric field may connect a plurality of isolated generators and a plurality of individual electrodes by a switching router to deliver a therapeutic electric field to the subject. Each generator can independently control the magnitude of the current, and the configuration of the switching router can be changed depending on the configuration of the electrode array and a preset operation sequence of pairs of electrode arrays.

For the tumor treating fields, the treatment planning system or the transducer array layout system may be used prior to the start of the treatment to determine the treatment plan in order to achieve maximum therapeutic effect. The system for delivering an electric field may receive prescribing information including the position of each electrode array, the operation sequence, the intensity of voltage and current of each pair of electrode arrays, the total number of treatment of electric field therapy, the total treatment time, the daily treatment time and the treatment frequency from the determined treatment plan to store in memory, and control each of the generators and the switching router by controller to initiate the treatment.

The temperature value measured by the temperature sensor thermally contacted with each of the individual electrodes may be sent to the system and, if the temperature is above the preset limit temperature, the current flowing to the electrode can be reduced or cut off. When the temperature falls below the limit temperature, it may switch to normal operation mode. By independently controlling the individual electrodes in the electrode array, the edge effect can be overcome and the reduction in the actual treatment time of delivering the electric field can be kept to a minimum.

According to an embodiment of the inventive concept, the transducer array layout system may be a transducer array layout system for a tumor treating fields based on body temperature regulation and absorption energy including an image classification unit that classifies organs and tumors in a patient's medical image containing organs and tumors, a property information setting unit that sets property information of each area classified by the image classification unit, a prescription information determination unit that determines the prescribed dose in consideration of an input tumor type and tumor status information, and determines the prescription information including the total number of treatments, the total treatment time, the daily treatment time and the treatment frequency of electric field therapy, a dose and temperature calculating unit that initially sets the number of electrodes, a position of the electrode, an application time of voltage and an intensity of voltage for each of the electrodes in consideration of the location, size and property information of each region classified by the image classification unit, and calculates a dose distribution and a temperature distribution in the body based on the initial setting, and an optimization unit that changes at least one of the number of electrodes, the position of the electrode and the application time and the intensity of voltage to optimize the dose and temperature distribution in the body so that the dose and temperature of each of the regions are satisfied to the preset dose standard and temperature standard.

The best treatment plan determined in the transducer array layout system may be input into the system for delivering an electric field, and the treatment may be performed. The electrode array may be attached to the skin of the subject based on the number of input electrodes and the position of the electrodes, and the order of operation and the duration of operation of the pair of electrode arrays and the intensity of voltage or current of the generator connected to each electrode may be set according to the intensity of voltage and the application time of each electrode.

An embodiment of the inventive concept, the transducer array layout system is a transducer array layout system of the tumor treating fields including a step to acquire a region of interest (ROI) and an organ at risk (OAR) information from a patient's medical image containing organs and tumors, a step to set the total shape and total area of the electrode array based on the acquired region of interest information, a step to set an area ratio occupied by the plurality of unit electrodes forming the electrode array in the total area of the electrode array, a step to repeatedly perform the step of setting the total shape and the total area and setting the area until the electric field delivered to the region of interest, and a step to derive a customized electrode array structure in which the electric field is optimized.

The best treatment plan determined in the transducer array layout system may be input into the system for delivering an electric field and the treatment may be performed. The electrode array may be attached to the skin of the subject according to the number of input electrodes and the position of the electrodes, and the order of operation and the duration of operation of the pair of electrode arrays, individual electrodes to be activated, and the intensity of voltage or current of the generator connected to each electrode may be set according to the intensity of voltage and the application time for each electrode.

Structure and Mode of Operation of System for Delivering an Electric Field

FIG. 2 shows a schematic view of a system for delivering an electric field. A control unit 100 may include an input/output interface (I/O interface) 106 receiving predetermined treatment plan data from the transducer array layout system, memory 108 storing data, and processor 107 controlling each generator 101 and a switching router 102 based on the stored data. The control unit may include a plurality of electrically isolated generators. The switching router including a switching router and an electrode switch is configured to select an electrode array and an electrode being connected to the generator. The switching router 102 may be configured as part of the control unit 100.

The control unit receives the prescription information, the position of the electrode array, and intensity of voltage or current of the individual electrode from the transducer array layout system via the I/O interface to store in the memory. The prescribing information may include the total number of the electric field therapy, the total treatment time, the daily treatment time, and the treatment frequency. In addition, a 3D model may be included to determine the exact attachment location of the electrode array.

The processor 107 of the controller may set a connection sequence and a duration of operation of the pairs of electrode arrays for cyclic operation according to data stored in the memory, and determine the mapping method between the electrodes of the active (in operation) electrode array pair and generators for each mode of operation and the initial value of voltage or current of each generator.

When treatment is initiated, processor 107 may repeat, for example, a first operation mode for a first duration of operation and a second operation mode for a second duration of operation, according to the sequence of the connection of the determined electrode array pair. When the first operation mode is started, the switching router may be controlled to select a first electrode array pair (a first electrode array and a second electrode array). Each electrode of the first electrode array and each electrode of the second electrode array may be connected to the generator according to a predetermined mapping configuration. The signal level may be determined according to intensity of voltage or current of the determined each generator, and current may be applied along a circuit connected to the mapped electrode in the first electrode array, the subject, and mapped electrode in the second electrode array to deliver an electric field into the subject body. After the determined first duration of operation, the processor may switch to the second operation mode. The switching router may be controlled to select a second electrode array pair (a third electrode array and a fourth electrode array). Each electrode of the third electrode array and each electrode of the fourth electrode array may be connected to the generator according to a predetermined mapping configuration. After the second duration of operation, the first operation mode may be switched. Two modes of operation are shown as an example, however a plurality of modes of operation may be repeatedly performed.

### Voltage Spike and Transient Current

The cyclic operation of the system for delivering an electric field requires a high switching speed of the switching router and the electrode switch that form the switching router. Considering that each mode of operation is about 1˜2 seconds, switching should be within a maximum of 100 ms. For fast response speed, the switching router and the electrode switch may use a FET switch. Each generator may experience a sudden change in load impedance as the switching and current change depending on the operation mode. This instant change may appear as a result of an unintentional spike or transient current added to a control current.

In an embodiment, in order to prevent the spike or the transient current that occurs at the moment when the switching router and the electrode switch are operated in the switching router, the switch may synchronize with the signal of the connected generator. FIG. 3 illustrates when the signal of the generator is desynchronized with the connected switch. If the switch is activated while the signal from the generator is applying voltage or current close to a non-zero peak, the load impedance of the generator may change abruptly to result in the spike or the transient current. FIGS. 4 and 5 illustrate a case in which a change in the mode of operation is synchronized with the signal of the generator.

In an embodiment, even if the FET switch is turned off when the switching router and the electrode switch are operated on the switching router, the impedance may be not infinite due to the parasitic capacity of the FET, and thus leakage current may be generated. FIG. 6 is an embodiment of a method for minimizing leakage current. Each FET switch 204 may be connected together with a relay switch 203. First, the relay switch 203 may be activated. When the relay switch is operating, a leakage current 208 may be generated. Within a short period of time after the relay switch 203 is activated, the FET switch 204 may be activated to apply the intended current and voltage 207. When switching to the next mode, turn off the FET switch first, and then turn off the relay switch to minimize the leakage current.

### Temperature Sensors and Thermal Management

Temperature management between the electrode and the skin is the most important aspect of the system for delivering an electric field, so the flow of current must be cut off when the limit temperature is exceeded in order to prevent the risk of skin burns. The electric current flowing between the electrode and the skin generates heat, and it is necessary to measure the temperature from an additional temperature sensor to prevent skin burns caused by this heat. When the measurement value of each temperature sensor exceeds a certain temperature (e.g., 41 degrees Celsius), there is an optimization method to avoid the risk of the subject being burned by pausing the system or changing the current waveform. It is also possible to initiate the shutdown of an operation mode instead of shutting down the entire system for delivering an electric field.

In an embodiment, a plurality of temperature sensors may be daisy chained to measure the temperature of the electrodes, so that the temperature of each electrode can be measured sequentially without additional circuit elements, and the measured temperature digital data may be transmitted through an interface cable. FIG. 7 is an embodiment of electrode array 103 including a temperature sensor unit connected to the control unit and the switching router and supporting a daisy chain. Each temperature sensor unit 300 may be in thermal contact with each of the electrode 104 so that the temperature of each electrode 104 can be measured. The best component for this purpose is the TMP144 from Texas Instruments. The component may include a temperature sensor, support UART communication, and support daisy-chaining. Thus, the temperature sensor unit 300 structure of daisy-chain may initiate temperature measurement through the controller 303 and acquire the temperature of each electrode 104 in each electrode array 103 sequentially in real time. The controller may receive the sequentially measured value of the temperature sensor and send the acquired temperature measurement value to the system for delivering an electric field.

The control unit may identify the hazardous electrode from the received temperature information and adjust the intensity of voltage and current of the generator connected to the electrode to prevent the temperature from rising above the limit temperature.

A key factor in thermal management is to determine which electrode in the electrode array has the highest temperature above the limit temperature. FIG. 8 shows the position information of the temperature sensor having the maximum temperature and the circuit that outputs the maximum temperature. It is assumed that the thermistor 400 in thermal contact with each electrode is the NTC type. The NTC thermistor 400 may have the characteristics that its resistance decreases as the temperature increases, and the output voltage decreases as the temperature increases. For example, if the temperatures of three thermistors Ra, Rb, and Rc shown in FIG. 8 are Ta, Tb, and Tc, and they satisfy the conditions of Ta>Tb>Tc, the output of each thermistor satisfies the relationship of Va<Vb<Vc. The voltage of each thermistor may be input to an Operational Amplifier (OP-Amp) 401 which is fed back through a diode 402 and a resistor. The current flowing through the bias resistor 403 may flow through a first diode 402a having the minimum voltage. At this time, a second diode 402b and a third diode 402c may be blocked. Thus, the Vout of the circuit may output a Va. A value minus the voltage corresponding to the diode voltage drop, Vd (=0.7 V) may output at a point 405a between a first amplifier 401a and the first diode 102a. On the other hand, the output of a second amplifier 402b may oscillate and therefore, a power supply Vcc of the second amplifier may output at a point 405b between the second amplifier and the second diode 405b. Similarly, the power supply Vcc of the amplifier may be output at a point 405c between a third amplifier 401c and the third diode 405c. In summary, the voltage output between the amplifier and the diode 405 is output Vt−Vd or Vcc so that the position information of the temperature sensor where the maximum temperature occurred may be obtained if the comparator and encoder are used.

### Configuration of the Switching Router

FIG. 9 is an embodiment of a method that converts the direction of the electric field into a unit of an electrode array. The switching router and the electrode switch may include a relay switch 500 of the SPDT (Single Pole Dual Terminals). The switching router and the electrode switch is configured to select each electrode in two arrays. The P directions of the switching router and the electrode switch may be synchronized with each other and selected by the electrode array, and the N directions of the switching routers may be synchronized with each other and selected by the electrode array. For example, one pair may be selected by selecting an electrode 1 from the electrode arrays A and B in the drawing and selecting an electrode 1 from the electrode arrays C and D in the N direction. The electrode 1 of the electrode array C and the electrode 1 of the electrode array D may be selected at the same time to apply voltage, and the electrode 1 of the electrode array A and the electrode 1 of the electrode array B may be selected and change the direction of the electric field to the left and right to apply to the subject.

FIG. 10 shows a direction of the electric field of the embodiment of FIG. 9. The region of interest 600 for the subject may be obtained through the input patient's medical image, and the direction of the electric field 601 and the intensity of voltage or current optimized for the region of interest 600 may be determined through the transducer array layout system. Referring to FIG. 10, the direction of the electric field 601 may be set in the transducer array layout system in order to apply electric field concentrating on the region of interest 600 and the plan of the transducer array layout system may be input to the control unit so that the optimized electric field is apply to the region of interest 601 to enhance the treatment effect.

Another embodiment of the inventive concept is to determine the direction of the electric field using a switch of the 1P4T method. FIG. 11 is an embodiment of a method that converts the direction of the electric field into units of an electrode array. The switching router and the electrode switch may include a 1P4T type relay switches 700(a) and 700(b) with one polarity that can select four directions. Each electrode switch is configured to select each electrode in the four electrode arrays 103. For example, each electrode array 103 attached to the subject 501 of the drawing may be divided into an electrode array of A, B, C, D and E, F, G, H. The system's P-direction switch 700(a) is configured to select an electrode from one of the electrode arrays A, B, C, and D, and an N-direction electrode switch 700(b) may connect to one of the electrode arrays E, F, G, and H. Therefore, it is possible to deliver an electric field in total of 16 different directions. However, in the actual treatment, the treatment plan should take into account the intensity, volume ratio and effect of the electric field delivered to the tumor.

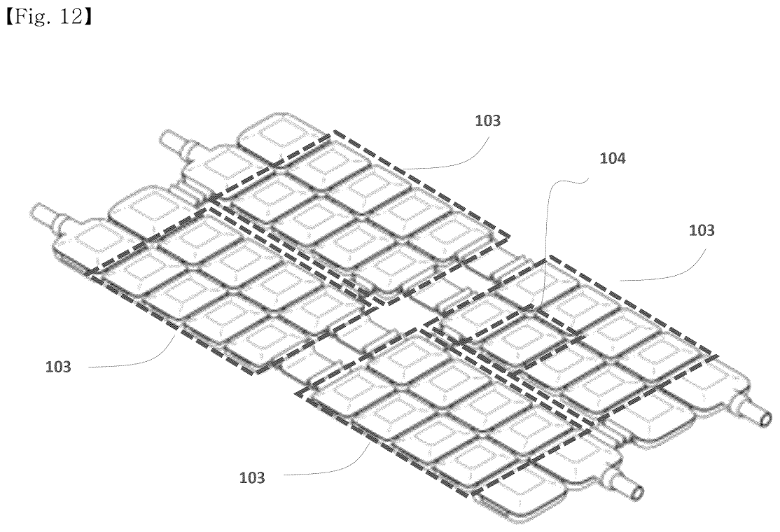

FIG. 12 is an embodiment of the electrode array group used in the embodiment of FIG. 11. Each electrode array 103 may consist of eight individual electrodes 104. An electrode array group may be attached to one side of the subject, and another group of electrode arrays may be attached to the other side of the subject. An electrode array group attached to one side may form electrode arrays A, B, C, and D, and another electrode array group attached to the other side may form electrode arrays of E, F, G, and H.

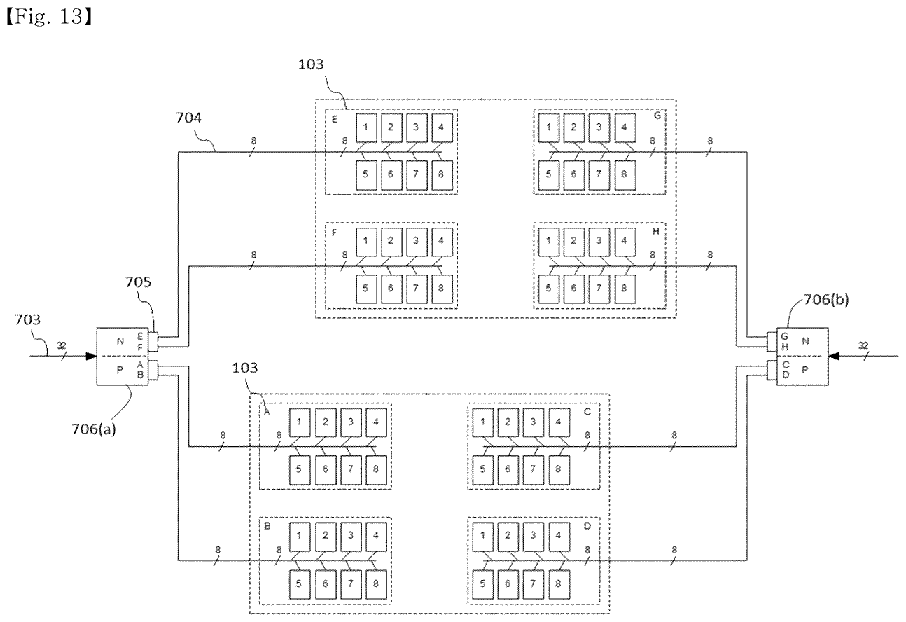

FIG. 13 is an embodiment of the configuration for administering the tumor treating fields using electrode array group used in the embodiment of FIG. 11. Each generator may be connected to the switching router 706 via connection line 703. One switch 706(a) may be connected to electrode arrays A, B, E, and F, and the other switch 706(b) may be connected to electrode arrays C, D, G, and H.

FIG. 14 shows a direction of the electric field in the embodiment of FIGS. 11 through 13. An optimized electric field centered on the subject's region of interest may be delivered. The system configured in the manner of 1P4T may connect the electrode of one of the electrode arrays A, B, C, and D to the electrode of the electrode arrays of E, F, G, and H. The selected electrode array may be activated 800 and the unselected electrode array may be disabled 801. Thus, possible cases of various directions can be formed in the region of interest 600, and the optimized direction and intensity of the electric field may be applied to the region of interest of the subject to increase the treatment effect.

On the other hand, the electrode sub-array 1302 may include at least one individual electrode and may include individual electrodes forming a row of the electrode array, or individual electrodes forming a column of the electrode array.

FIG. 15 shows an embodiment of controlling each electrode individually. Each electrode may be controlled by an individual generator and can be selected by switching router and the electrode switch. For example, a switching router and the electrode switch may activate 902a all electrodes 103 in a single electrode array 104 to apply an electric field. The electrodes connected to each generator and the switching router may be selected by each electrode array, as well as electrodes from other electrode arrays to apply a configuration voltage.

The tumor treating fields may obtain the position of the region of interest through the medical image, determine and the direction of the electric field and the intensity of voltage or current optimized for the region of interest through the transducer array layout system. Therefore, through this example which can give a rotating electric field, it can be applied in several directions centered on the region of interest. In addition, the present embodiment may select electrodes for each electrode array via a switching router, however select other electrodes within the electrode array. Thus, based on the selected electrode, it is possible to apply electric fields in various directions.

FIG. 16 is an embodiment viewed from above when an electric field is applied in centered on a tumor of the embodiment of FIG. 15. The transducer array layout system is configured to determine the direction of the electric field in order to apply the optimal electric field to the region of interest. Therefore, the electric field may be applied in a total of four or more directions, once based on a first direction that connects the electrodes closest to the tumor, and then in the direction closest to 90-degree, 45-degree, and 135-degree. The determined direction of the electric field may be applied in the order of 0-degree, 90-degrees, 45-degrees, and 135-degrees, and the direction of the electric field in any order may change through the switching router to apply it to the subject's region of interest thereby increasing the treatment effect.

FIG. 17 shows a direction of the electric field controlled by each electrode in the embodiment of FIG. 15. Since each electrode can be controlled compared to the previous example, the electric field can be applied in more flexible directions. For each electrode, the electrode can be activated 950 or deactivated 951. Therefore, the direction of the electric field can be applied in a more diverse way, thereby minimizing the angle of ineffectiveness of the electric field tumor treatment.

### Optimization of System for Delivering an Electric Field

According to an embodiment of the inventive concept, the transducer array layout system may be a transducer array layout system of a tumor treating fields based on body temperature regulation and absorption energy including an image classification unit that classifies organs and tumors in a patient's medical image containing organs and tumors, a property information setting unit that sets property information of each area classified by the image classification unit, a prescription information determination unit that determines the prescribed dose in consideration of an input tumor type and tumor status information, and determines the prescription information including the total number of treatments, the total treatment time, the daily treatment time and the treatment frequency of electric field therapy, a dose and temperature calculating unit that initially sets the number of electrodes, a position of the electrode, an application time of voltage and an intensity of voltage for each of the electrodes in consideration of the location, size and property information of each region classified by the image classification unit, and calculates a dose distribution and a temperature distribution in the body based on the initial setting, and an optimization unit that changes at least one of the number of electrodes, the position of the electrode and the application time and the intensity of voltage to optimize the dose and temperature distribution in the body so that the dose and temperature of each of the regions are satisfied to the preset dose standard and temperature standard.

FIG. 18 shows the progress of treatment using the transducer array layout system of a tumor treating fields based on the body temperature regulation and the absorption energy. First, the medical image of the subject may be acquired and input into the transducer array layout system S120. Based on the medical image of the subject, the major organ and the region of interest may be set S121, and number of electrodes, position of the electrodes, and application time and intensity of voltage or current of each electrode may be initially set S122. Based on this, absorption dose and temperature distribution in the body may be calculated S123. The calculated absorption dose and temperature distribution in the body may be evaluated S124 to determine whether the electric field is minimized in the major organs and optimized in the region of interest, or whether temperature of the major organs, the region of interest, and skin do not rise to dangerous temperatures S125. If the evaluated results are not optimized, at least one of the position and number of electrodes, the intensity of voltage and current of each electrode, and application time may be adjusted and recalculated S131, S123 and evaluated S124. If the results are determined to be optimized, the transducer array layout system may output the result about the intensity of voltage and current applied to each electrode S126. Based on the output results, the corresponding data may be input into the control unit S127. The control unit may receive the result of the transducer array layout system and determine the possible operating cases of the switching router by considering the configuration of the electrodes and the switching router S128. The treatment may begin by applying the electric field in order or in any sequence of the possible cases of the determined direction of electric field S129, and the control unit may measure the electrode temperature of the electrode array in real time and regulate current or voltage of the problematic electrode S130. By using the transducer array layout system, the optimized electric field may be applied to the region of interest and the minimum electric field to the major organ, and the temperature of the skin or the region of interest and major organs may be calculated in advance to reduce the risk of side effects and dramatically increase the treatment effect.

The best treatment plan determined in the transducer array layout system may be input to the system for delivering an electric field tumor electric field and the treatment may be performed. According to the number of input electrodes and the position of the electrodes, the electrode array may be attached to the skin of the subject, and the order of operation and the duration of operation of the pair of electrode arrays and the intensity of voltage or current of the generator connected to each electrode may be set according to the intensity of voltage and the application time of each electrode.

An embodiment of the inventive concept, the transducer array layout system may be a transducer array layout system of the tumor treating fields including a step to acquire a region of interest and an organ at risk information from a patient's medical image containing organs and tumors, a step to set the total shape and total area of the electrode array based on the acquired region of interest information, a step to set an area ratio occupied by the plurality of unit electrodes forming the electrode array in the total area of the electrode array, a step to repeatedly perform the step of setting the total shape and the total area and setting the area until the electric field delivered to the region of interest, and a step to derive a customized electrode array structure in which the electric field is optimized.

The intensity of the electric field delivered to the subject's region of interest may increase as increasing the area of the electrode, but the average intensity of the electric field delivered to the region of interest may be the same when a certain area is reached. Therefore, it is necessary to deliver the maximum electric field to the area of interest and a safe electric field to the main organ. FIG. 19 is a block diagram of the process of treatment by determining the position of the electrode to be activated using the transducer array layout system and intensity of voltage or current of the activated electrode. First, the subject's 3D medical image data may be input into the transducer array layout system S140. An information about the region of interest and major organs may be obtained from the input data of the subject S141. Based on the acquired region of interest and the position information of the major organs, activated electrodes or deactivated electrodes in the electrode array may be determined S142. Next, an intensity and a ratio of area of each electrode occupied in the activated electrode array may be set S143, and whether the intensity of the electric field delivered to the region of interest and the major organs is optimized overall or not may be evaluated S144. If it is determined to be optimized, the result about the position of the activated electrode and the intensity of voltage or current applied to each electrode may be output S145, and it may input into the control unit based on the obtained result S146. Based on the results received from the transducer array layout system, a control method may be determined in consideration of components of the electrode and the switching router.

FIG. 20 shows an embodiment of selecting an electrode in the embodiment of FIG. 15 based on the calculated result of the transducer array layout system and applying an electric field optimized for the region of interest. In order to apply the optimized electric field to the region of interest 600 and the major organ 620, the electrode may be selected to resemble the shape of the tumor. The electrodes selected to resemble shape of the tumor 950 may be activated, while the unselected electrodes may be inactivated 951. The intensity of voltage or current calculated in the transducer array layout system may be then applied through the activated electrode.

Although the invention has been described with reference to a specific embodiment, this description is not meant to be construed in a limiting sense. On the contrary, various modifications of the disclosed embodiments will become apparent to those skilled in the art upon reference to the description of the invention without modification of technical spirit or essential feature of the inventive concept. For example, those skilled in the art may change the material, size, etc. of each component according to the field of application, or combine or replace the embodiments disclosed and perform them in a form that is not clearly disclosed in the embodiment of the inventive concept, but this is also not beyond the scope of the inventive concept. Therefore, the embodiments described above are exemplary in all respects and should not be understood as being limited, and it should be said that these variations embodiments are included in the technical ideas described in claims of the inventive concept.
1000: System for delivering an electric field
1100: A plurality of generators
1102: Generator
1202: Switching router
1204: Electrode switch
1300: Electrode array
1302: Electrode sub-array
1304: Individual electrode
1400: Controller
105: Connection line
Switch on/off signal
Signal of mode 1 electrode
Signal of mode 2 electrode
Relay switch signal
FET switch signal
304: Connector
P-direction switch
N-direction switch
P-direction connection line
N-direction connection line
702: Connector
705: Connector

1. A system for delivering an electric field to a region of interest in a three-dimensional subject, the system comprising:
a plurality of generators configured to provide the electric field;
a plurality of electrode sub-arrays, each configured to deliver alternating current power, generated by the plurality of generators, to the subject, each electrode sub-array including a portion of individual electrodes comprising a plurality of individual electrodes;
a switching router configured to one-to-one connect the plurality of generators and the plurality of electrode sub-arrays; and
a controller configured to control the plurality of generators, the plurality of electrode sub-arrays, and the switching router.

2. The system of claim 1, wherein the controller is configured to independently provide adjustable current to each of the electrode sub-arrays to prevent excessive current from being applied to any particular electrode sub-array.

3. The system of claim 1, wherein the controller is configured to independently control a magnitude of voltage or current and to independently control a duty cycle for applying the voltage or current for each of the plurality of generators.

4. The system of claim 1, wherein the controller is configured to determine a mapping method between the plurality of generators and the plurality of electrode sub-arrays, and to control the switching router based on the mapping method.

5. The system of claim 4, wherein the switching router is configured to establish a mapping connection between the plurality of generators and the plurality of sub-arrays.

6. The system of claim 5, wherein the switching router comprises a relay switch.

7. The system of claim 6, wherein the switching router further comprises a field-effect transistor (FET) connected in series with the relay switch.

8. The system of claim 1, further comprising a temperature sensor configured to detect a temperature rise of each individual electrode,
wherein, the controller is configured to, for each electrode sub-array where a temperature rise is detected, independently reduce a duty cycle for applying the current or to decrease a magnitude of voltage or current provided by a generator connected to the respective electrode sub-array.

9. The system of claim 1, wherein the controller is configured to receive electrode layout information from an external transducer array layout system, the electrode layout information including a position of the electrode array, a selection of the electrode sub-arrays, and an intensity of voltage or current for each electrode sub-array.

10. The system of claim 9, wherein the position of the electrode array is represented on a 3D model of the subject.

11. The system of claim 9, wherein the controller is configured to, based on the electrode layout information, determine a mapping method between the plurality of generators and the plurality of electrode sub-arrays, and to control the switching router accordingly.

12. The system of claim 11, wherein the controller is configured to, based on the electrode layout information, set a connection order and an operation duration of the electrode sub-arrays, and to determine a mapping method and an initial value of voltage or current for each of the plurality of generators for each mode of operation.

13. A system for delivering an electric field to a region of interest in a three-dimensional subject, the system comprising:
a generator is configured to provide the electric field;
an electrode sub-array comprising a portion of individual electrodes from the electrode array and configured to deliver alternating current power, generated by the generator, to the subject;
a switching router configured to connect the generator and the electrode sub-array;
at least one electrode switch configured to connect the electrode sub-array and at least one of the individual electrodes; and
a controller configured to control the generator, the electrode sub-array, the switching router, and the at least one electrode switch.

14. The system of claim 13, wherein the controller is configured to select at least one electrode sub-array and provide adjustable current to the selected electrode sub-array such that a dose distribution delivered to the region of interest matches a location, shape, and size of the region of interest.

15. The system of claim 13, wherein the controller is configured to control a magnitude of voltage or current of the generator, and to control a duty cycle for applying voltage or current.

16. The system of claim 13, wherein the switching router and at least one electrode switch comprise a relay switch, and a field-effect transistor (FET) connected in series with the relay switch.

17. The system of claim 13, wherein each individual electrode of the electrode sub-array further comprises a temperature sensor configured to detect a temperature rise, and
the controller is configured to, when a temperature rise is detected, reduce a duty cycle for applying current or decrease a magnitude of voltage or current of the generator connected to the corresponding electrode sub-array.

18. The system of claim 13, wherein the controller is configured to receive electrode operation information from an external transducer array layout system, the electrode operation information including (i) selection status of the connection, (ii) an operation time, and (iii) an operation power for each of the electrode sub-arrays and a plurality of external electrode sub-arrays, and to sequentially repeat application of operation power based on the connection selection and the operation time for each of the electrode sub-arrays and the external electrode sub-arrays.

19. A method for delivering an electric field to a region of interest in a three-dimensional subject, comprising:
configuring at least one electrode sub-array from an electrode array including a plurality of individual electrodes; and
sequentially and repeatedly delivering the electric field via at least one pair of configured electrode sub-arrays.

20. The method of claim 19, wherein each of the electrode sub-arrays is configured to vary an electric field intensity or an electric field application time.
